(19) United States
(12) Patent Application Publication
(10) Pub. No.: US 2024/0182525 A1
(43) Pub. Date: Jun. 6, 2024

(54) NOVEL BICYCLIC COMPOUND, OPTICAL ISOMER THEREOF OR PHARMACEUTICALLY ACCEPTABLE SALT THEREOF, USE THEREOF, AND PREPARATION METHOD THEREFOR

(71) Applicant: KOREARESEARCH INSTITUTE OF BIOSCIENCE AND BIOTECHNOLOGY, Daejeon (KR)

(72) Inventors: Jong Seog AHN, Daejeon (KR); Jun Pil JANG, Daejeon (KR); Jae-Hyuk JANG, Daejeon (KR); Sung-Kyun KO, Daejeon (KR); Min Cheol KWON, Daejeon (KR); Gui Ja HWANG, Daejeon (KR); Jae-Won LEE, Daejeon (KR)

(73) Assignee: KOREARESEARCH INSTITUTE OF BIOSCIENCE AND BIOTECHNOLOGY, Daejeon (KR)

(21) Appl. No.: 18/282,160

(22) PCT Filed: Mar. 14, 2022
(86) PCT No.: PCT/KR2022/003517

§ 371 Date: Sep. 14, 2023

(30) Foreign Application Priority Data
Mar. 15, 2021 (KR) 10-2021-0033075

(51) Int. Cl. C07K 7/56; A61P 3/04; A61P 19/08; C12N 1/20; A61K 38/00; C12R 1/465

(52) U.S. Cl. CPC C07K 7/56; A61P 3/04; A61P 19/08; C12N 1/205; A61K 38/00; C12R 2001/465

(57) ABSTRACT

The present invention provides a compound represented by chemical formula 1, an optical isomer thereof or a pharmaceutically acceptable salt thereof, and a pharmaceutical composition for preventing or treating Wnt/β-catenin signaling-associated diseases, comprising same.

### TECHNICAL FIELD

The present invention relates to a novel bicyclic compound, an optical isomer thereof, or a pharmaceutically acceptable salt thereof, and more particularly, to a composition for preventing, improving or treating Wnt/β-catenin signaling-associated diseases by having a function of promoting the activity of the Wnt/β-catenin signaling pathway.

### BACKGROUND ART

Physiologically active substances derived from microorganisms have generally been sources of antibacterial, antifungal, and anticancer drugs, and further, have been developed as new drugs for the treatment of various diseases, or have been the template for new drug development. Representative examples of antibiotics derived from microorganisms include amphotericin, erythromycin, streptomycin, tetracycline and vancomycin. In addition, in 2003, daptomycin isolated from the actinomycete Streptomyces was approved by the US Food and Drug Administration (FDA) as a next-generation antibiotic. Further, as a pharmaceutical drug for diet and calorie control, lipstatin, found in the bacterium Streptomyces toxytricini, is marketed as a lipase inhibitor called Orlistat, with minor chemical modifications. As described above, research on physiologically active substances derived from bacteria, particularly actinomycetes, is very important in pharmaceuticals and drug development. It is necessary to select microorganisms that are structurally different from existing substances and produce beneficial physiologically active substances, and to search and discover novel compounds produced by the microorganisms.

Wnt signaling cascades are signaling molecules involved in various effects of embryonic development, including cell fate specification, polarity and cell migration. The Wnt signaling pathway is activated by Wnt ligands, and the Wnt growth factor family includes 10 or more genes identified in mouse and at least 7 genes identified in humans. Members of the Wnt family of signaling molecules mediate many important short- and long-range patterning processes during invertebrate and vertebrate development. The Wnt signaling pathway is known for its role in the inductive interactions that regulate growth and differentiation, and likely also plays important roles in the homeostatic maintenance of post-embryonic tissue integrity. Wnt stabilizes cytoplasmic P-catenin, which stimulates the expression of genes including c-myc, c jun, fra-1, and cyclin D1.

In addition, there are many genetic disorders caused by mutations in Wnt signaling components. Examples of genetic disorder include Alzheimer's disease, osteoarthritis, colorectal polyps, bone density and ocular vascular deficiency (osteoporosis-pseudoglioma syndrome, OPPG), familial exudative vitreous retinopathy, retinal neovascularization, and the like.

Activation of the Wnt signaling pathway in adipocytes blocks the differentiation of adipocytes by suppressing the expression of adipocyte-specific transcription factors, peroxisome proliferator-activated receptor-γ (PPARγ) and CCAAT/enhancer-binding protein α (C/EBPα). Thus, research to explore substances that reduce fat accumulation and generation of adipocytes through inhibition of differentiation into adipocytes is being actively conducted.

In addition, it is known that the mutation of low-density lipoprotein receptor-related protein 5 (Lrp5), one of the Wnt protein receptors, regulates bone mass, indicating that the Wnt signaling pathway is related to bones, and it has been reported that osteoporosis pseudoglioma syndrome (OPPG), a representative genetic disease caused by a decrease in bone density, is observed when a loss-of-function mutation occurs in the Lrp5 gene in humans (inactivation of the Wnt/β-catenin signaling pathway). It has been reported that a gain of function mutation (G171V mutation) in the Lrp5 gene in humans (activation of the Wnt/β-catenin signaling pathway) causes an increase in bone mass. Therefore, the relevance of the Wnt/β-catenin signaling pathway to the bone growth and bone density control has been actively studied, which has been spotlighted as an effective and safe drug treatment target for the treatment of osteoporosis.

Control of cell signaling by the Wnt signaling pathway is important for neuronal circuit formation. The Wnt pathway regulates, among other things, axon pathfinding, dendrite development, and synaptic assembly in neural tissue. Through different receptors, the Wnt pathway activates and/or modulates various signaling pathways and other processes, leading to local changes on the cytoskeleton or global cellular changes related to nuclear function.

Therefore, the present inventors found that novel bicyclic compounds derived from soil actinomycetes and derivatives thereof not only directly affect the Wnt/β-catenin signaling pathway, but also have effects in treating and improving Wnt signaling-associated diseases, and completed the present invention.

### DISCLOSURE

### Technical Problem

An object of the present invention is to provide a novel bicyclic compound represented by Chemical Formula 1, an optical isomer thereof, or a pharmaceutically acceptable salt thereof having an effect of promoting the activity of the Wnt/β-catenin signaling pathway.

In addition, another object of the present invention is to provide a method for preparing the novel bicyclic compound represented by Chemical Formula 1, an optical isomer thereof, or a pharmaceutically acceptable salt thereof.

Further, still another object of the present invention is to provide a Wnt/β-catenin signaling activator comprising the novel bicyclic compound represented by Chemical Formula 1, an optical isomer thereof, or a pharmaceutically acceptable salt thereof.

Further, still another object of the present invention is to provide a pharmaceutical composition for preventing or treating Wnt/β-catenin signaling-associated diseases, comprising: the novel bicyclic compound represented by Chemical Formula 1, an optical isomer thereof, or a pharmaceutically acceptable salt thereof.

Further, still another object of the present invention is to provide a food composition for preventing or improving Wnt/β-catenin signaling-associated diseases, comprising the novel bicyclic compound represented by Chemical Formula 1, an optical isomer thereof, or a pharmaceutically acceptable salt thereof.

In addition, still another object of the present invention is to provide a method for treating Wnt/β-catenin signaling-associated diseases comprising: administering a therapeutically effective amount of the novel bicyclic compound represented by Chemical Formula 1, an optical isomer thereof, or a pharmaceutically acceptable salt thereof to a subject in need thereof.

Further, still another object of the present invention is to provide use of the novel bicyclic compound represented by Chemical Formula 1, an optical isomer thereof, or a pharmaceutically acceptable salt thereof for the manufacture of a medicament for the treatment of Wnt/β-catenin signaling-associated diseases.

In addition, still another object of the present invention is to provide a novel Streptomyces rapamycinicus 17A011 strain (Accession Number: KCTC 14890BP).

### Technical Solution

Terms used in the present application are only used to describe specific embodiments and are not intended to limit the present invention. Singular expressions include plural expressions unless the context clearly indicates otherwise. In the present specification, terms such as “comprise”, “have”, and the like are intended to designate the presence of features, steps, structures, or combinations thereof described in the specification and it should not be understood as precluding the possibility of the presence or addition of one or more other features, steps, structures, or combinations thereof.

Further, unless defined otherwise, all terms used herein, including technical or scientific terms, have the same meaning as commonly understood by one of ordinary skill in the art to which the present invention belongs. Terms such as those defined in commonly used dictionaries should be interpreted as having a meaning consistent with the meaning in the context of the related art, and unless explicitly defined in the present application, it is not to be construed in an idealized or overly formal sense.

In the present invention, “Cx-y” means having a carbon number of x or more and y or less.

In the present invention, with respect to the term “single bond”, the phrase that X₁ is a single bond in Chemical Formula 1 of the present invention does not mean that X₁ connects X₃ and a phenyl group with a linker such as a carbon atom or an oxygen atom but means a direct covalent bond between the phenyl group and X₃. As a specific example, in Ph-X₁-X₃-X₂-Ph, when X₁ and X₂ are single bonds and X₃ is an oxygen atom (O), a Ph-O-Ph bond structure may be formed.

As used herein, the term “halogen” means a substituent selected from fluorine (F), chloro (Cl), bromo (Br) and iodo (I).

As used herein, the term “substituted” refers to a moiety having a substituent group that replaces hydrogen on one or more carbons in the main chain. “Substitution” or “substituted with” depends on whether such substitution is permissible for the substituted atom and substituent, and it is defined to include the implicit conditions that leads to stable compounds by substitution, for example, compounds that are not naturally modified by rearrangement, cyclization, elimination, and the like.

As used herein, the term “C₁₋₆ alkyl” means a C₁₋₆ straight or branched chain saturated hydrocarbon such as methyl, ethyl, propyl, isopropyl, butyl, pentyl, or hexyl. Preferred alkyl groups include about 1, 2, 3, 4, 5 or 6 carbon atoms in the chain. Side chain means that at least one lower alkyl group, such as methyl, ethyl or propyl, is attached to the linear alkyl chain. Term “lower alkyl” means a group having from about 1 to about 4 carbon atoms in the chain that may be straight or branched. The “alkyl” may be unsubstituted or optionally substituted by one or more substituents that may be the same or different, each substituent being hydroxyl (—OH), halogen atom, C₁₋₆ alkyl, or oxo (═O), etc. Preferably, the substituted alkyl may be an alkyl having 1 to 3 carbon atoms in which at least one hydroxyl group is substituted.

As used herein, the term “C₂₋₆ alkene” refers to a straight or branched chain hydrocarbon chain containing one or more double bonds. Alkenes may have 2 to 6 carbon atoms, and may also be lower alkenes having 2 to 4 carbon atoms. The alkene may be referred to as alkenyl in the present invention. The alkene or alkenyl represents, as an example, 2 to 6 carbon atoms in the alkene chain, and in other words, the alkene chain is selected from the group consisting of ethene (ethenyl), propene (propenyl) (for example, propen-1-yl, propen-2-yl, propen-3-yl), butene (butenyl) (for example, buten-1-yl, buten-2-yl, buten-3-yl, buten-4-yl), 1-methyl-propen-1-yl, 2-methyl-propen-1-yl, 1-ethyl-ethen-1-yl, 2-methyl-propen-3-yl, buta-1,3-dienyl, buta-1,2-dienyl and buta-1,2-dien-4-yl. Typical alkenyl groups include, but are not limited to, ethenyl, propenyl, butenyl, pentenyl and hexenyl, and the like. In addition, “alkene” may be unsubstituted or optionally substituted by one or more substituents that may be the same or different, each substituent being hydroxyl (—OH), halogen atom, C₁₋₆ alkyl, or oxo (═O), etc. Preferably, the alkene may be substituted with one or more hydroxyl groups. The substituted alkene in the present invention may be preferably (Z)-prop-1-ene-1,2-diol or (E)-prop-1-ene-1,2-diol, substituted with two hydroxyl groups.

As used herein, “C₃₋₈ heterocycloalkyl” means a saturated monocyclic and polycyclic heterocycle containing 1 to 3 oxygen (O) atoms or a ring structure in which two or more rings share at least one pair of carbon atoms. The heterocycloalkyl includes oxirane (oxiranyl), oxetane (oxetanyl), morpholinyl, thiethanyl, dioxolane, pyrrolidinyl, piperidinyl, tetrahydrofuranyl, tetrahydrothiophenyl, tetrahydropyranyl, tetrahydrothiopyranyl, and the like, and may preferably be oxirane or dioxolane, but is not limited thereto. In addition, “heterocycloalkyl” may be unsubstituted or optionally substituted by one or more substituents that may be the same or different, each substituent being hydroxyl (—OH), a halogen atom, C₁₋₆ alkyl or oxo (═O), etc. Preferably, the heterocycloalkyl may be independently substituted with one or more of alkyl, oxo, and the like.

As used herein, “C₃₋₈ heterocycloalkene” refers to a ring structure in which two or more rings share at least one pair of carbon atoms or monocyclic and polycyclic heterocycles having at least one double bond while containing 1 to 3 oxygen (O) atoms, wherein the rings are not aromatic. The heterocycloalkene includes oxirene, oxetene, dihydrofuran, and the like, and may preferably be oxirene, and the scope of the present invention is not limited thereto. In addition, “heterocycloalkene” may be unsubstituted or optionally substituted by one or more substituents that may be the same or different, each substituent being hydroxyl (—OH), a halogen atom, C₁₋₆ alkyl or oxo (═O), etc. Preferably the substituted heterocycloalkene may be substituted with one or more alkyls.

In addition, terms and abbreviations used in the present specification have original meanings thereof unless otherwise defined.

To solve the above problems, the present inventors found that a bicyclic compound represented by the following Chemical Formula 1, an optical isomer thereof, or a pharmaceutically acceptable salt thereof, isolated and identified from a fraction of the culture of Streptomyces rapamycinicus 17A011 strain (Accession Number: KCTC 14890BP) promotes Wnt/β-catenin signaling pathway activity and is effective in preventing, improving, and treating Wnt/β-catenin signaling-associated diseases, and completed the present invention.

Hereinafter, each of the novel bicyclic compounds of the present invention, a preparation method, and use thereof will be described in detail.

### Novel Bicyclic Compound, Optical Isomer Thereof or Pharmaceutically Acceptable Salt Thereof

In one aspect of the present invention, there is provided a compound represented by the following Chemical Formula 1, an optical isomer thereof, or a pharmaceutically acceptable salt thereof:

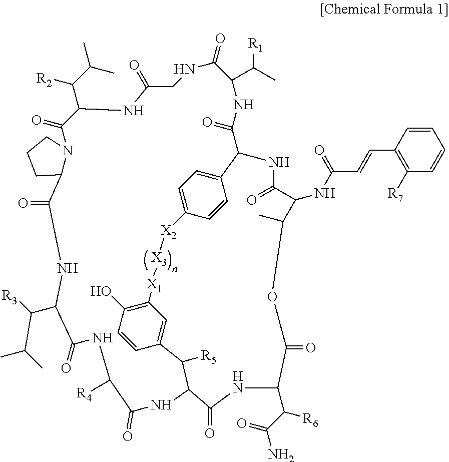

in Chemical Formula 1 above,

R₁, R₂, R₃, R₄, R₅ and R₆ are the same as or different from each other, and are each independently a hydrogen atom or hydroxyl (—OH);

R₇ is a substituted or unsubstituted C₁₋₆ alkyl, a substituted or unsubstituted C₂₋₆ alkene, a substituted or unsubstituted C₃₋₈ heterocycloalkyl containing at least one oxygen atom (O) as a heteroatom, or a substituted or unsubstituted C₃₋₈ heterocycloalkene containing at least one oxygen atom (O) as a heteroatom (wherein at least one H of the substituted C₁₋₆ alkyl, the substituted C₂₋₆ alkene, the substituted C₃₋₈ heterocycloalkyl, or the substituted C₃₋₈ heterocycloalkene may each independently be substituted with hydroxyl (—OH), a halogen atom, C₁₋₆ alkyl or oxo (═O));

n is an integer of either 0 or 1;

when n is 0, X₁ and X₂ are the same as or different from each other, and are each independently a hydrogen atom or hydroxyl (—OH); and

when n is 1, X₁ and X₂ are each independently a single bond, and X₃ is —O—.

In an embodiment, the compound represented by Chemical Formula 1 may be a compound represented by the following Chemical Formula 2, an optical isomer thereof, or a pharmaceutically acceptable salt thereof:

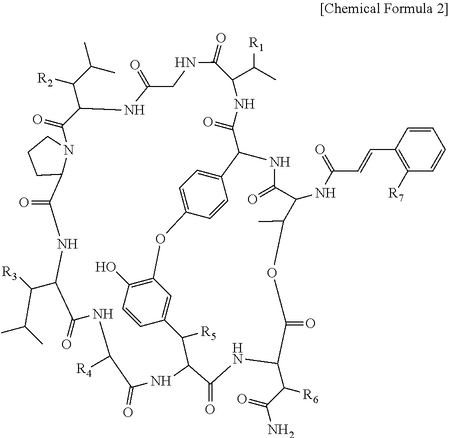

in Chemical Formula 2 above,

R₁, R₂, R₃, R₄, R₅ and R₆ are the same as or different from each other, and are each independently a hydrogen atom or hydroxyl (—OH); and

R₇ is a substituted or unsubstituted C₁₋₆ alkyl, a substituted or unsubstituted C₂₋₆ alkene, a substituted or unsubstituted C₃₋₈ heterocycloalkyl containing at least one oxygen atom (O) as a heteroatom, or a substituted or unsubstituted C₃₋₈ heterocycloalkene containing at least one oxygen atom (O) as a heteroatom (wherein at least one H of the substituted C₁₋₆ alkyl, the substituted C₂₋₆ alkene, the substituted C₃₋₈ heterocycloalkyl, or the substituted C₃₋₈ heterocycloalkene may each independently be substituted with hydroxyl (—OH), a halogen atom, C₁₋₆ alkyl or oxo (═O)).

In an embodiment, the compound represented by Chemical Formula 1 may be a compound represented by the following Chemical Formula 3, an optical isomer thereof, or a pharmaceutically acceptable salt thereof:

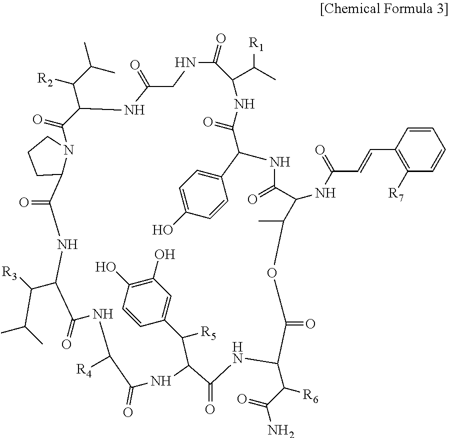

in Chemical Formula 3 above,

R₁, R₂, R₃, R₄, R₅ and R₆ are the same as or different from each other, and are each independently a hydrogen atom or hydroxyl (—OH);

R₇ is a substituted or unsubstituted C₁₋₆ alkyl, a substituted or unsubstituted C₂₋₆ alkene, a substituted or unsubstituted C₃₋₈ heterocycloalkyl containing at least one oxygen atom (O) as a heteroatom, or a substituted or unsubstituted C₃₋₈ heterocycloalkene containing at least one oxygen atom (O) as a heteroatom (wherein at least one H of the substituted C₁₋₆ alkyl, the substituted C₂₋₆ alkene, the substituted C₃₋₈ heterocycloalkyl, or the substituted C₃₋₈ heterocycloalkene may each independently be substituted with hydroxyl (—OH), a halogen atom, C₁₋₆ alkyl or oxo (═O)).

In an embodiment, in the Chemical Formula 1 or 2 of the present invention, R₁, R₂, R₄, and R₅ may each independently be hydroxyl (—OH); R₃ and R₆ may each independently be a hydrogen atom.

In an embodiment, in the Chemical Formula 3 of the present invention, R₁, R₂, R₄, and R₅ may each independently be hydroxyl (—OH); R₃ and R₆ may each independently be a hydrogen atom.

In an embodiment, in Chemical Formula 1 or 2 above, R₇ may be C₁₋₄ alkyl in which at least one H is each independently substituted with hydroxyl (—OH), C₂₋₄ alkene in which at least one H is each independently substituted with hydroxyl (—OH), C₃₋₅ heterocycloalkyl containing at least one oxygen atom (O) as a heteroatom in which at least one H is each independently substituted with C₁₋₃ alkyl, oxo (═O), or both, or C₃₋₅ heterocycloalkene containing at least one oxygen atom (O) as a heteroatom in which at least one H is each independently substituted with C₁₋₃ alkyl.

Preferably, in the Chemical Formula 1 or 2, R₇ may be C₁₋₄ alkyl substituted with at least two hydroxyls (—OH), C₂₋₄ alkene substituted with at least two hydroxyls (—OH), C₃₋₅ heterocycloalkyl containing at least one oxygen atom (O) substituted with C₁₋₃ alkyl as a heteroatom, C₃₋₅ heterocycloalkyl containing at least one oxygen atom (O) substituted with C₁₋₃ alkyl and oxo (═O) as a heteroatom, or C₃₋₅ heterocycloalkene containing one oxygen atom (O) substituted with one C₁₋₃ alkyl as a heteroatom.

In an embodiment, in the Chemical Formula 3, R₇ may be C₁₋₄ alkyl in which one or more Hs are each independently substituted with hydroxyl (—OH), preferably C₁₋₄ alkyl substituted with at least two hydroxyls (—OH).

In an embodiment, in the Chemical Formula 1 or 2, R₇ is propane, propene, oxirane, oxirene or dioxolane, wherein the propane, propene, oxirane, oxirene and dioxolane may be unsubstituted, or may each independently be substituted with hydroxyl (—OH) or C₁₋₃ alkyl. Preferably, R₇ may be propane substituted with two hydroxyl (—OH) groups, propene substituted with two hydroxyl (—OH) groups, oxirane substituted with C₁₋₃ alkyl, oxirene substituted with C₁₋₃ alkyl, or dioxolane substituted with oxo (═O) and C₁₋₃ alkyl.

In an embodiment, in the Chemical Formula 3, R₇ may be propane, preferably propane substituted with two hydroxyl (—OH) groups.

In an embodiment, in the Chemical Formula 1 or 2, R_{7 may be}

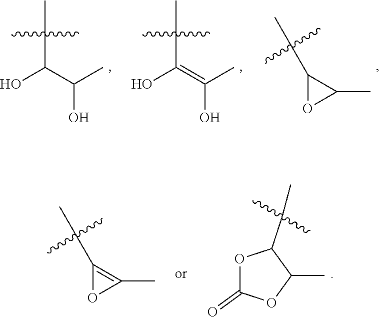

In an embodiment, in the Chemical Formula 3, R₇ may be

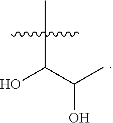 [SMILES: *C(O)C(C)O]

According to an embodiment of the present invention, the compound represented by Chemical Formula 1, an optical isomer thereof or a pharmaceutically acceptable salt thereof may be any one selected from the group consisting of the following Chemical Formulas 1-1, 1-2, 1-3, 1-4, 1-5 and 1-6:

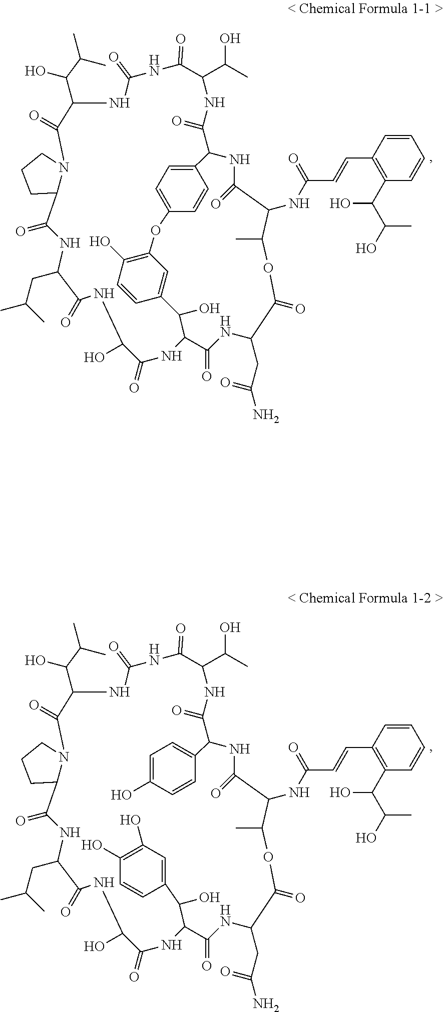 [SMILES: CC(C)CC1NC(=O)C2CCCN2C(=O)C(C(O)C(C)C)NC(=O)NC(=O)C(C(C)O)NC(=O)C2NC(=O)C(NC(=O)/C=C/c3ccccc3C(O)C(C)O)C(C)OC(=O)C(CC(N)=O)NC(=O)C(NC(=O)C(O)NC1=O)C(O)c1ccc(O)c(c1)Oc1ccc2cc1 ; CC(C)CC1NC(=O)C2CCCN2C(=O)C(C(O)C(C)C)NC(=O)NC(=O)C(C(C)O)NC(=O)C(c2ccc(O)cc2)NC(=O)C(NC(=O)/C=C/c2ccccc2C(O)C(C)O)C(C)OC(=O)C(CC(N)=O)NC(=O)C(C(O)c2ccc(O)c(O)c2)NC(=O)C(O)NC1=O]

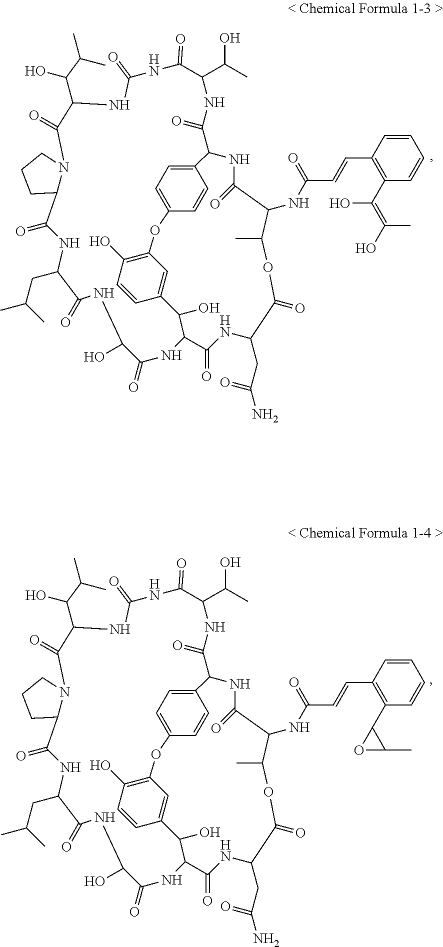 [SMILES: C/C(O)=C(/O)c1ccccc1/C=C/C(=O)NC1C(=O)NC2C(=O)NC(C(C)O)C(=O)NC(=O)NC(C(O)C(C)C)C(=O)N3CCCC3C(=O)NC(CC(C)C)C(=O)NC(O)C(=O)NC(C(=O)NC(CC(N)=O)C(=O)OC1C)C(O)c1ccc(O)c(c1)Oc1ccc2cc1 ; CC(C)CC1NC(=O)C2CCCN2C(=O)C(C(O)C(C)C)NC(=O)NC(=O)C(C(C)O)NC(=O)C2NC(=O)C(NC(=O)/C=C/c3ccccc3C3OC3C)C(C)OC(=O)C(CC(N)=O)NC(=O)C(NC(=O)C(O)NC1=O)C(O)c1ccc(O)c(c1)Oc1ccc2cc1]

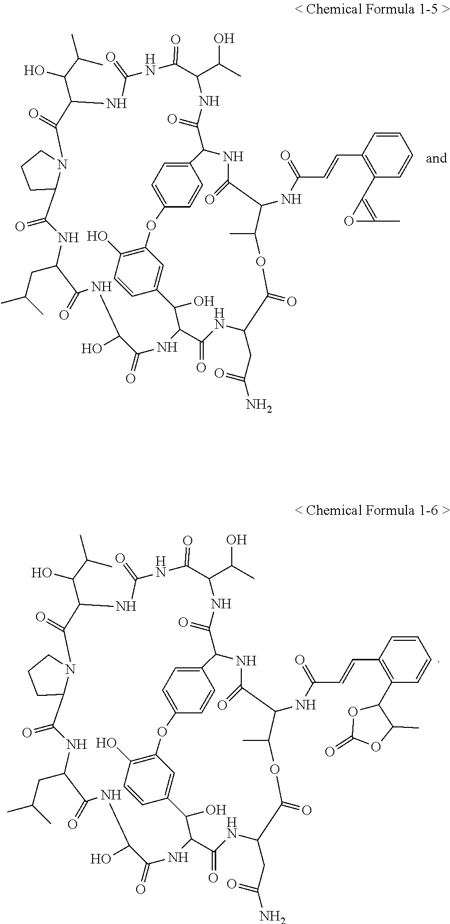 [SMILES: CC1=C(c2ccccc2/C=C/C(=O)NC2C(=O)NC3C(=O)NC(C(C)O)C(=O)NC(=O)NC(C(O)C(C)C)C(=O)N4CCCC4C(=O)NC(CC(C)C)C(=O)NC(O)C(=O)NC(C(=O)NC(CC(N)=O)C(=O)OC2C)C(O)c2ccc(O)c(c2)Oc2ccc3cc2)O1 ; CC(C)CC1NC(=O)C2CCCN2C(=O)C(C(O)C(C)C)NC(=O)NC(=O)C(C(C)O)NC(=O)C2NC(=O)C(NC(=O)/C=C/c3ccccc3C3OC(=O)OC3C)C(C)OC(=O)C(CC(N)=O)NC(=O)C(NC(=O)C(O)NC1=O)C(O)c1ccc(O)c(c1)Oc1ccc2cc1]

According to an embodiment of the present invention, the compound of Chemical Formula 2 may be any one selected from the group consisting of the Chemical Formulas 1-1, 1-3, 1-4, 1-5 and 1-6.

According to an embodiment of the present invention, the compound represented by Chemical Formula 3 may be represented by the Chemical Formula 1-2.

In the present invention, the compound represented by Chemical Formula 1 above, an optical isomer thereof, or a pharmaceutically acceptable salt thereof may be isolated from Streptomyces rapamycinicus 17A011 strain (Accession Number: KCTC 14890BP), and the strain may have the gene sequence of SEQ ID NO: 1 as follows:

[SEQ ID NO: 1]
GTGCATGGCTGTCGTCAGCTCGTGTCGTGAGATGTTGGGTTAAGTCCCGC
AACGAGCGCAACCCTTGTTCTGTGTTGCCAGCATGCCTTTCGGGGTGATG
GGGACTCACAGGAGACTGCCGGGGTCAACTCGGAGGAAGGTGGGGACGAC
GTCAAGTCATCATGCCCCTTATGTCTTGGGCTGCACACGTGCTACAATGG
CCGGTACAATGAGCTGCGAAGCCGTGAGGTGGAGCGAATCTCAAAAAGCC
GGTCTCAGTTCGGATTGGGGTCTGCAACTCGACCCCATGAAGTCGGAGTC
GCTAGTAATCGCAGATCAGCATTGCTGCGGTGAATACGTTCCCGGGCCTT
GTACACACCGCCCGTCACGTCACGAAAGTCGGTAACACCCGAAGCCGGTG
GCCCAACCCTTGTGGAGGGAGCTGTCGAAGTGGGACTGGCGAT.

The compound represented by Chemical Formula 1 of the present invention includes not only salts thereof, but also optical isomers capable of being prepared from the salts by conventional methods.

In the present invention, as the “salt” of the compound represented by Chemical Formula 1, an acid addition salt formed by a free acid is useful. The acid addition salts are obtained from inorganic acids such as hydrochloric acid, nitric acid, phosphoric acid, sulfuric acid, hydrobromic acid, hydroiodic acid, nitrous acid, phosphorous acid, and the like, aliphatic mono- and dicarboxylates, phenyl-substituted alkanoates, hydroxy alkanoates and alkanedioates, aromatic acids, non-toxic organic acids such as aliphatic and aromatic sulfonic acids, and the like, organic acids such as acetate, benzoic acid, citric acid, lactic acid, maleic acid, gluconic acid, methanesulfonic acid, 4-toluenesulfonic acid, tartaric acid, fumaric acid, and the like. Examples of these salt include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitro benzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, βhydroxybutyrate, glycolate, malate, tartrate, methanesulfonate, propanesulfonate, naphthalene-1-sulfonate, naphthalene-2-sulfonate, mandelate, and the like.

In addition, the acid addition salt may be prepared by a conventional method, for example, may be prepared by dissolving the compound represented by Chemical Formula 1 in an organic solvent such as methanol, ethanol, acetone, methylene chloride, acetonitrile, or the like, and adding an organic acid or an inorganic acid to form a precipitate, followed by filtering and drying the resulting precipitate, or may be prepared by distilling a solvent and excess acid under reduced pressure, followed by drying and crystallizing the product in an organic solvent.

Further, a metal salt may be prepared using a base. The alkali metal salt or alkaline earth metal salt is obtained, for example, by dissolving the compound in an excess alkali metal hydroxide or alkaline earth metal hydroxide solution, and filtering an insoluble compound salt, followed by evaporating and drying the filtrate. Here, it is pharmaceutically suitable to prepare a sodium, potassium or calcium salt as the metal salt. In addition, the corresponding salt is obtained by reacting an alkali metal or alkaline earth metal salt with a suitable negative salt (for example, silver nitrate).

In the present invention, the term “optical isomer” refers to two stereoisomers of a compound that have non-overlapping mirror images, and diastereomers are stereoisomers having two or more asymmetric centers in which molecules of the centers are not mirror images of each other.

As used herein, “the pharmaceutically acceptable salt” means a salt commonly used in the pharmaceutical industry, and may include, for example, inorganic ion salts prepared from calcium, potassium, sodium, magnesium, and the like, inorganic acid salts prepared from hydrochloric acid, nitric acid, phosphoric acid, bromic acid, iodic acid, perchloric acid, and sulfuric acid, and the like; organic acid salts prepared from acetic acid, trifluoroacetic acid, citric acid, maleic acid, succinic acid, oxalic acid, benzoic acid, tartaric acid, fumaric acid, manderic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like; sulfonic acid salts prepared from methanesulfonic acid, ethanesulfonic acid, benzenesulfonic acid, p-toluenesulfonic acid, naphthalenesulfonic acid, and the like; amino acid salts prepared from glycine, arginine, lysine, and the like; and amine salts prepared with trimethylamine, triethylamine, ammonia, pyridine, picoline, and the like, but the types of salts referred to in the present invention are not limited by these listed salts.

Specifically, it was found from Examples that the bicyclic compound represented by Chemical Formula 1 above, an optical isomer thereof, or a pharmaceutically acceptable salt thereof had an effect of promoting the activity of the Wnt/β-catenin signaling pathway by separation from a fraction of the culture of Streptomyces rapamycinicus 17A011 (Accession Number: KCTC 14890BP), which may be appreciated to have a therapeutic effect for Wnt/β-catenin signaling pathway-associated diseases (FIGS. 1 to 17). Thus, the bicyclic compound represented by Chemical Formula 1 of the present invention may be used as an active ingredient of a drug for preventing, improving, or treating Wnt/β-catenin signaling pathway-associated diseases.

### Method for Preparing Novel Bicyclic Compound, Optical Isomer Thereof or Pharmaceutically Acceptable Salt Thereof

In one aspect of the present invention, in order to obtain the compound represented by Chemical Formula 1, a fraction of the culture of Streptomyces rapamycinicus 17A011 (Accession Number: KCTC 14890BP) may be prepared by a preparation method comprising the following steps, but the method is not limited thereto:

1) isolating and culturing Streptomyces rapamycinicus 17A011 (Accession Number: KCTC 14890BP) to obtain a strain culture;

2) extracting the strain culture obtained in Step 1) above with an organic solvent to obtain an extract; and

3) fractionating the extract obtained in Step 2 above using an organic solvent.

In the present invention, in Step 1), the strain may be cultured in a medium containing a source of nutrients capable of being used by common microorganisms. As the source of nutrients, known nutrient sources conventionally used for culturing actinomycetes may be used. For example, a carbon source may be glucose, starch syrup, dextrin, starch, molasses, animal oil, vegetable oil, etc., and a nitrogen source may be wheat bran, soybean meal, wheat, malt, cottonseed gourd, fish gourd, cornsteeplicker, broth, yeast extract, ammonium sulfate, sodium nitrate, urea, etc. If necessary, table salt, potassium, magnesium, cobalt, chlorine, phosphoric acid, sulfuric acid and other inorganic salts promoting ion generation may be added. As a culture method, shaking culture or stationary culture is possible under aerobic conditions. The culture temperature is slightly different depending on the conditions when cultured under the above respective conditions, but generally may be at 20° C. to 37° C., or may also be 25° C. to 30° C., but is not limited thereto. In the present invention, a more preferable culture medium may be YMG medium containing glucose, soluble starch, yeast extract, malt extract, or calcium carbonate.

The organic solvent may be water, C₁ to C₄ lower alcohol, hexane, chloroform, methylene chloride, ethyl acetate, acetone, acetonitrile, or a combination thereof.

In Step 2) above, the bicyclic compound according to the present invention may be present not only in the culture media of the strain but also in the cell part. Therefore, an organic solvent is added to the culture media and cells of the strain to extract active ingredients from the culture solution and cells, and then the obtained extract is concentrated by evaporation under reduced pressure. At this time, it is preferable to use ethyl acetate as the organic solvent.

In addition, in Step 3), the organic solvent may be water, ethanol, methanol, or a combination thereof, preferably a mixed solvent of water and methanol. Specifically, elution may be performed using methanol/water (8:2-10/0, v/v) as a mixed solvent while gradually increasing the methanol concentration to achieve fractionation.

In the present invention, fractions of the culture of Streptomyces rapamycinicus 17A011 (Accession Number: KCTC 14890BP) may be fractionated by thin layer chromatography or liquid chromatography to separate the compound represented by Chemical Formula 1, specifically compounds represented by Chemical Formulas 1-1 to 1-6.

In the present invention, the fractionating in Step 3) may be performed by performing column chromatography on the extract obtained in Step 2 using a mixed solvent of methanol and water, followed by purification by high-performance liquid chromatography.

The column chromatography and high-performance liquid chromatography techniques refer to chromatography techniques in which a mobile phase is a liquid, and is performed in a column filled with a stationary phase or on a plane to which a stationary phase is attached. The fraction of the culture of Streptomyces rapamycinicus 17A011 (Accession Number: KCTC 14890BP) of the present invention is not limited as long as it is a solvent fraction. As the mobile phase, an organic solvent such as water, hexane, methanol, ethanol, acetonitrile, acetone, chloroform, dichloromethane, ethyl acetate, or the like, may be used alone or in combination, and the stationary phase may be silica gel, Diaion HP-20, RP-18 or Sephadex LH-20, but the mobile phase and the stationary phase are not limited thereto.

In an embodiment of the present invention, the present inventors extracted and isolated the bicyclic compound of the present invention from a terrestrial soil actinomycete, Streptomyces rapamycinicus 17A011 (Accession Number: KCTC 14890BP) strain, and confirmed the isolated compound.

### Pharmaceutical Composition or Food Composition Comprising Novel Bicyclic Compound, Optical Isomer Thereof or Pharmaceutically Acceptable Salt Thereof

The present invention also provides a pharmaceutical composition for preventing or treating Wnt/β-catenin signaling-associated diseases, comprising: the compound represented by Chemical Formula 1, an optical isomer thereof, or a pharmaceutically acceptable salt thereof.

Further, the present invention provides a Wnt/β-catenin signaling activator comprising the compound represented by Chemical Formula 1, an optical isomer thereof, or a pharmaceutically acceptable salt thereof.

The compounds according to the present invention may be used for the treatment of Wnt/β-catenin signaling-associated diseases by promoting the activity of the Wnt/β-catenin signaling pathway.

The Wnt/β-catenin signaling-associated diseases may include neurodegenerative diseases, eye diseases, bone diseases, periodontal diseases, otosclerosis (ear sclerosis), wound healing, oral mucositis, gastrointestinal mucositis, craniofacial defects, hair loss diseases, and metabolic diseases.

The bone disease may include bone defect, osteoporosis, osteoarthrosis, osteogenesis imperfecta, bone defect, osteoporotic fracture, diabetic fracture, nonunion fracture, osteogenesis imperfecta, osteomalacia and resulting fractures, bone dysplasia, degenerative bone disease, oncolytic bone disease, Paget's disease, metabolic bone disease, leukemia, multiple myeloma, myeloma, fibrous osteodysplasia, aplastic bone disease, osteonecrosis, rickets, or malocclusion.

The bicyclic compound according to the present invention is useful for treating bone diseases by increasing alkaline phosphatase (ALP) activity (FIGS. 14 to 17).

In the present invention, the metabolic disease may be obesity, dyslipidemia, fatty liver, diabetes, hyperlipidemia, hypercholesterolemia, arteriosclerosis, myocardial infarction, cerebral infarction, sarcopenia, hyperinsulinemia, and myocardial infarction.

In the present invention, the neurodegenerative disease may be Parkinson's disease, stroke, spinal cord injury, ischemic brain disease, epilepsy, Alzheimer's disease, dementia, depression, bipolar disorder, and schizophrenia.

In the present invention, the eye disease may be wet macular degeneration, dry age-related macular degeneration, geographic atrophy, diabetic retinopathy, diabetic macular edema, retinal detachment, retinal degeneration, retinal vein occlusion, retinopathy of prematurity, retinitis pigmentosa, retinopathy, Leber congenital amaurosis, and glaucoma.

For administration, the pharmaceutical composition of the present invention may further comprise at least one pharmaceutically acceptable carrier in addition to the compound represented by Chemical Formula 1 or a pharmaceutically acceptable salt thereof. The pharmaceutically acceptable carrier may be saline, sterile water, Ringer's solution, buffered saline, dextrose solution, maltodextrin solution, glycerol, ethanol, or a mixture of one or more of these components, and, if necessary, may contain other conventional additives such as antioxidants, buffers, bacteriostatic agents, and the like. Further, the composition may be formulated into injectable formulations such as aqueous solutions, suspensions, emulsions, and the like, pills, capsules, granules or tablets by further adding diluents, dispersants, surfactants, binders and lubricants thereof. Accordingly, the pharmaceutical composition of the present invention may be a patch, liquid, pill, capsule, granule, tablet, suppository, or the like. These preparations may be prepared by conventional methods used for formulation in the art or methods disclosed in the document [see, Remington's Pharmaceutical Science (latest edition), Mack Publishing Company, Easton PA], and formulated into various preparations depending on respective diseases or components.

The composition of the present invention is administered in a pharmaceutically effective amount. In the present invention, the term “pharmaceutically effective amount” means an amount that is sufficient to treat a disease at a reasonable benefit/risk ratio applicable to medical treatment without causing side effects, wherein the effective dose level may be determined depending on factors including the patient's health condition, the type and severity of the disease, drug activity, drug sensitivity, administration method, administration time, the route of administration and the excretion rate, the duration of treatment, drugs used in combination or concurrently, and other factors well known in the medical field. The composition of the present invention may be administered as an individual therapeutic agent or in combination with other therapeutic agents, may be administered sequentially or simultaneously with conventional therapeutic agents, and may be administered singly or in multiple doses. In consideration of all of the above factors, it is important to administer an amount capable of obtaining the maximum effect with the minimum amount without side effects, which may be easily determined by those skilled in the art. The daily dosage of the compound represented by Chemical Formula 1 of the present invention may be about 0.01 to 1000 mg/kg, preferably 0.1 to 100 mg/kg, and the compound represented by Chemical Formula 1 of the present invention may be administered in an amount divided into one to several times a day.

The term “administration” of the present invention means introducing a predetermined substance into a patient by an appropriate method, and the composition may be administered through any general route as long as the composition is able to reach the target tissue. In addition, the pharmaceutical composition of the present invention may be administered by any device capable of transporting an active substance to a target tissue. For example, the administration may be oral administration, intrathecal administration, intraperitoneal administration, intravenous administration, intramuscular administration, subcutaneous administration, intradermal administration, topical administration, intranasal administration, intrapulmonary administration, intrarectal administration, inner ear administration, endometrial administration, sublingual administration, or intracerebrovascular injection, but is not limited thereto.

Solid preparations for oral administration include tablets, pills, powders, granules, capsules, and the like, and these solid preparations are formulated by mixing at least one excipient in the composition, such as starch, calcium carbonate, sucrose, lactose, gelatin, etc. In addition, lubricants such as magnesium stearate and talc may be used in addition to simple excipients.

Examples of oral liquid preparation may include suspensions, solutions for internal use, emulsions, syrups, and the like. Various excipients such as wetting agents, sweeteners, aromatics, preservatives, and the like, may be included in addition to water and liquid paraffin that are commonly used simple diluents.

Preparations for parenteral administration may include sterilized aqueous solutions, non-aqueous solutions, suspensions, emulsions, freeze-dried preparations, suppositories. As the non-aqueous solvents and the suspensions, propylene glycol, polyethylene glycol, vegetable oils such as olive oil, and injectable esters such as ethyl oleate, and the like, may be used. As the base of the suppository, witepsol, macrogol, Tween 61, cacao butter, laurin butter, glycerogelatin and the like, may be used. Meanwhile, conventional additives such as solubilizers, isotonic agents, suspending agents, emulsifiers, stabilizers, preservatives, and the like, may be contained in the injection.

The composition may be administered through any general route as long as the composition is able to reach the target tissue, and for example, the composition may be administered through intradermal injection, intravein injection, intraperitoneal injection, intravitreal injection, subcutaneous injection using an osmotic pump, or the like.

The pharmaceutical composition of the present invention may further comprise at least one active ingredient exhibiting the same or similar efficacy in addition to the compound represented by Chemical Formula 1 or a pharmaceutically acceptable salt thereof.

In still another aspect, the present invention provides a food composition for preventing or improving Wnt/β-catenin signaling-associated diseases, comprising the compound represented by Chemical Formula 1, an optical isomer thereof, or a food-acceptable salt thereof.

In addition to the active ingredient, the composition may comprise food-acceptable food additives.

The term “food supplement additive” used herein refers to a component capable of being added to food in an auxiliary manner, and may be appropriately selected and used by those skilled in the art as being added to prepare a health functional food of each formulation. Examples of the food supplement additive include various nutrients, vitamins, minerals (electrolytes), flavors such as synthetic flavors, natural flavors, and the like, colorants and fillers, pectic acid and salts thereof, alginic acid and salts thereof, organic acids, protective colloidal thickeners, pH regulators, stabilizers, preservatives, glycerin, alcohol, carbonation agents used in carbonated beverages, and the like, but the types of food additives of the present invention are not limited by these examples.

The food composition of the present invention may include health functional food. The term “health functional food” used herein refers to food prepared and processed in the form of tablets, capsules, powders, granules, liquids and pills using raw materials or ingredients having useful functionalities for the human body. Here, ‘functionality’ means to obtain useful effects for health purposes, such as nutrient control, physiological action, and the like, on the structure and function of the human body. The health functional food of the present invention may be prepared by a method commonly used in the art, and may be prepared by adding raw materials and components commonly added in the art during the preparation. In addition, the formulation of the health functional food may also be manufactured without limitation as long as the formulation is recognized as a health functional food. The composition for food of the present invention may be prepared in various types of formulations, and unlike general drugs, may be made from food to have the advantage of not having side effects that may occur when taking drugs for a long time and may have excellent portability. Thus, the health functional food of the present invention may be ingested as an adjuvant to enhance the effect of anticancer drugs.

As used herein, the term “prevention” refers to any activity that inhibits cancer formation or delays the onset of cancer by administration of the composition.

As used herein, the term “improvement” refers to any action that at least reduces the severity of a parameter associated with the condition being treated, for example a symptom.

In the present invention, “treatment” refers to all activities in which the symptoms of the disease are improved or beneficially changed by administration of the composition.

### Therapeutic Use of Wnt/β-Catenin Signaling-Associated Diseases, Treatment Method, and Use Thereof in the Manufacture of Medicaments For Treatment

The present invention provides a method for treating Wnt/β-catenin signaling-associated diseases comprising: administering a therapeutically effective amount of the compound represented by Chemical Formula 1, an optical isomer thereof, or a pharmaceutically acceptable salt thereof to a subject in need thereof.

The subject in need thereof refers to mammals, including humans, and includes mammals such as humans, monkeys, cows, horses, dogs, cats, rabbits, rats, and mice.

As used herein, the term “therapeutically effective amount” refers to an amount of the novel salt or pharmaceutical composition containing the same effective for preventing or treating Wnt/β-catenin signaling-associated diseases, such as an amount of the novel salt administered to the subject to be treated, which may include all of the amounts of the pharmaceutical composition comprising the above-described salts that prevent occurrence or recurrence, alleviate symptoms, impair direct or indirect pathological consequences, prevent metastasis, slow the rate of progression, alleviate or temporarily alleviate the condition, or improve the prognosis, of Wnt/β-catenin signaling-associated diseases. In other words, the therapeutically effective amount may be interpreted as encompassing all doses at which symptoms of Wnt/β-catenin signaling-associated diseases are improved or cured by the pharmaceutical composition.

The method for preventing or treating Wnt/β-catenin signaling-associated diseases of the present invention also includes not only dealing with the diseases themselves prior to the onset of symptoms, but also inhibiting or avoiding symptoms thereof, by administering the above-described salt or pharmaceutical compositions comprising the salt. In the management of diseases, a prophylactic or therapeutic dose of a particular active ingredient will vary depending on the nature and severity of the disease or condition and the route by which the active ingredient is administered. The dose and frequency of dose will vary depending on the individual patient's age, weight and response. A suitable dosage regimen may be readily selected by those skilled in the art who take these factors for granted. In addition, the method of treating Wnt/β-catenin signaling-associated diseases using the pharmaceutical composition of the present invention may further comprise the administration of a therapeutically effective amount of an additional active agent useful for treating a disease together with the above-described salt, wherein additional active agent may exhibit synergistic or auxiliary effects with the above-described salt which is an active ingredient according to the present invention.

Further, the present invention provides use of the compound represented by Chemical Formula 1, an optical isomer or a pharmaceutically acceptable salt thereof for the manufacture of medicaments for the treatment of Wnt/β-catenin signaling-associated diseases. The above-described salt for the manufacture of medicaments may be mixed with acceptable adjuvants, diluents, carriers, and the like, and may be prepared as a combined preparation with other active agents to have a synergistic effect of the active ingredients.

The present invention also provides a pharmaceutical composition for use in the prevention or treatment of Wnt/β-catenin signaling-associated diseases, comprising: the compound represented by Chemical Formula 1, an optical isomer thereof, or a pharmaceutically acceptable salt thereof.

Matters mentioned in the uses, compositions and treatment methods of the present disclosure are applied equally as long as they do not contradict each other.

### Advantageous Effects

The novel bicyclic compound according to the present invention may activate the Wnt/β-catenin signaling pathway to be used to treat Wnt/β-catenin signaling-associated diseases.

In addition, the compound of the present invention may minimize side effects as a natural product, and may be easily extracted and purified since culturable actinomycetes are used, which is very advantageous in view of economic efficiency.

### DESCRIPTION OF DRAWINGS

FIG. 1 shows TopFLASH luciferase assay measurement protocol.

FIG. 2 shows the Wnt activity evaluation results of the bicyclic compound represented by Chemical Formula 1 and Wnt3a of the present invention.

FIG. 3 shows cytotoxicity test results in 3T3-L1 cells for the treatment of the bicyclic compound represented by Chemical Formula 1-1 alone (L) and the combined treatment of Wnt3a and Chemical Formula 1-1 (Wnt3a) of the present invention.

FIG. 4 shows the Wnt activity evaluation (TopFLASH assay) results through treatment with the bicyclic compound represented by Chemical Formula 1-1 of the present invention.

FIG. 5 shows the Wnt activity evaluation (TopFLASH assay) results through the combined treatment of the bicyclic compound represented by Chemical Formula 1-1 and Wnt3a of the present invention.

FIG. 6 shows changes in Wnt3a signaling protein for the bicyclic compound represented by Chemical Formula 1-1 of the present invention.

FIG. 7 quantitatively shows changes in Wnt3a signaling protein for the bicyclic compound represented by Chemical Formula 1-1 of the present invention.

FIG. 8 shows experimental results of the differentiation inhibitory effect on adipogenic differentiation cells (T3-L1 cells) for the bicyclic compound represented by Chemical Formula 1-1 of the present invention.

FIG. 9 shows experimental results of the differentiation inhibitory effect on adipogenic differentiation cells (3T3-L1 cells) for the combined treatment of the bicyclic compound represented by Chemical Formula 1-1 and wnt3a.

FIG. 10 shows experimental results of changes in differentiation-associated protein in adipogenic differentiation cells (3T3-L1 cells) for the bicyclic compound represented by Chemical Formula 1-1 of the present invention.

FIG. 11 shows experimental results of changes in differentiation-associated mRNA in adipogenic differentiation cells (3T3-L1 cells) for the bicyclic compound represented by Chemical Formula 1-1 of the present invention.

FIG. 12 shows the efficacy evaluation results of the bicyclic compound represented by Chemical Formula 1-1 of the present invention in suppressing obesity in mice using a high-calorie feed.

FIG. 13 shows measurement results for obesity and liver damage factors in mouse serum using a high-calorie feed for the bicyclic compound of Chemical Formula 1-1 of the present invention.

FIG. 14 shows cytotoxicity test results in MC3T3-E1 cells depending on the concentration of the bicyclic compound represented by Chemical Formula 1-1 of the present invention.

FIG. 15 shows ALP activity results according to the treatment of the bicyclic compound represented by Chemical Formula 1-1 of the present invention.

FIG. 16 shows the analysis results of alkaline phosphatase (ALP) activity of the bicyclic compound represented by Chemical Formula 1-1 of the present invention in MC3T3-E1 cells.

FIG. 17 shows Western blotting results of confirming the expression of bone differentiation-involved genes for the bicyclic compound represented by Chemical Formula 1-1 of the present invention.

### BEST MODE

Hereinafter, exemplary embodiments will be described in detail.

However, the following Examples are only provided to illustrate the present invention, and the content of the present invention is not limited to these Examples.

### EXAMPLE 1

### Identification and Culture of Streptomyces rapamycinicus 17A011 Strain

(1) Deposit, Isolation and Identification of Streptomyces rapamycinicus 17A011 Strain

Soil samples were collected from Ochang, Cheongju, South Korea. As a result of 16S rRNA gene sequence analysis, the 17A011 strain was found to be most closely related to the Streptomyces rapamycinicus gene (99.79% homology, GenBank Accession No. KP209440.1). Thus, strain 17A011 was named Streptomyces rapamycinicus 17A011 and used in subsequent culture experiments.

(2) Culture of Streptomyces rapamycinicus 17A011 Strain

In order to culture the present actinomycete strain, a medium containing a source of nutrients commonly used by microorganisms was prepared. The used crude medium and fermentation medium for actinomycetes was YMG medium containing 10 g glucose, 20 g soluble starch, 5 g yeast extract, 5 g malt extract, and 0.5 g calcium carbonate per 1 L of distilled water. A 1000 ml Erlenmeyer flask containing 250 ml of the seed medium was sterilized at 121° C. for 15 minutes, then Streptomyces rapamycinicus 17A011 strain was inoculated with a platinum loop from a test tube for slant culture and cultured with shaking for 3 days to form a seed culture. Each of 40 Erlenmeyer flasks with a capacity of 1,000 ml containing 250 ml of fermentation medium (10 L in total) was inoculated with 3 ml of the seed culture media, followed by culturing with shaking for 7 days at 28° C.

### EXAMPLE 2

### Isolation and Purification of Bicyclic Compounds from Streptomyces rapamycinicus 17A011 Strain

The culture media (10 L) of Streptomyces rapamycinicus 17A011 strain cultured in Example 1 was centrifuged and extracted with 9 L of ethyl acetate, and the obtained extract was concentrated by evaporation under reduced pressure using a vacuum dryer. This concentrate (4.5 g) was adsorbed on ODS RP-18 and subjected to ODS RP-18 flash column chromatography, wherein a total of 10 fractions were obtained by eluting with methanol/water (8:2-10/0, v/v) as a mixed solvent while gradually increasing the methanol concentration. Here, fraction No. 7 (440 mg) containing Chemical Formulas 1-1, 1-2, 1-3, 1-4, 1-5 and 1-6 was concentrated under reduced pressure, followed by high performance liquid chromatography (column: Cosmosil C18, length 25 mm, diameter 10 mm) to perform elution under conditions of an elution flow rate of 3 ml/min using 0.05% formic acid-containing acetonitrile and water (40:60) as a solvent, thereby preparing novel bicyclic compounds exhibiting UV absorption peaks of 210 nm and 260 nm.

### EXAMPLE 3

### Structural Analysis of Bicyclic Compound from Streptomyces rapamycinicus 17A011 Strain

The molecular weight and molecular formula of the bicyclic compounds according to the present invention prepared from the culture media of Streptomyces rapamycinicus 17A011 strain were determined using an electrospray ionization mass spectrometer (ESIMS). In addition, nuclear magnetic resonance (NMR) analysis (Bruker AVANCE HD 800 NMR spectrometer) was performed to obtain ¹H NMR, ¹³C NMR, Correlation Spectroscopy (COSY), ¹H-Detected heteronuclear Multiple-Quantum Coherence (HMQC), Heteronuclear Multiple-Bond Coherence (HMBC), Distortionless Enhancement by Polarization Transfer (DEPT) and Nuclear Overhauser effect spectroscopy (NOESY) spectra, and molecular structures of the compounds were determined.

The measurement results are shown in Table 1 below. Materials isolated from the culture media of Streptomyces rapamycinicus 17A011 strain were identified as novel bicyclic compounds of the following Chemical Formulas 1-1, 1-2, 1-3, 1-4, 1-5 and 1-6, and results thereof are shown in Table 1 below:

TABLE 1

| Compound | Structure | NMR/MS data |
|---|---|---|
| Chemical Formula 1-1 | 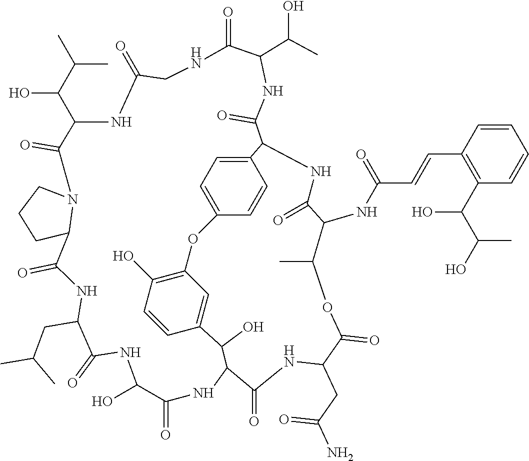 [SMILES: CC(C)CC1NC(=O)C2CCCN2C(=O)C(C(O)C(C)C)NC(=O)CNC(=O)C(C(C)O)NC(=O)C2NC(=O)C(NC(=O)/C=C/c3ccccc3C(O)C(C)O)C(C)OC(=O)C(CC(N)=O)NC(=O)C(NC(=O)C(O)NC1=O)C(O)c1ccc(O)c(c1)Oc1ccc2cc1] | ¹H NMR (800 MHz, Pyr) δ 11.01 (d, J = 9.3 Hz, 1H), 9.98 (d, J = 3.8 Hz, 1H), 9.49 (d, J = 7.4 Hz, 1H), 9.41 (d, J = 9.6 Hz, 1H), 9.15 (d, J = 10.8 Hz, 2H), 8.61-8.57 (m, 1H), 8.53 (s, 1H), 8.38 (d, J = 2.9 Hz, 1H), 8.10 (d, J = 7.8 Hz, 1H), 8.02 (dd, J = 8.5, 2.0 Hz, 1H), 7.95 (s, 1H), 7.72 (dd, J = 8.1, 2.0 Hz, 1H), 7.67 (d, J = 8.4 Hz, 1H), 7.51 (dd, J = 8.1, 2.4 Hz, 1H), 7.32 (d, J = 8.4 Hz, 1H), 7.26 (d, J = 7.8 Hz, 1H), 7.24 (t, J = 6.8 Hz, 2H), 7.12 (dd, J = 8.4, 2.3 Hz, 1H), 7.09 (d, J = 1.8 Hz, 1H), 6.74 (d, J = 9.3 Hz, 2H), 6.58 (t, J = 7.4 Hz, 1H), 6.00 (q, J = 6.9 Hz, 1H), 5.83 (d, J = 4.5 Hz, 1H), 5.76 (d, J = 3.1 Hz, 1H), 5.71 (d, J = 3.9 Hz, 1H), 5.57 (d, J = 10.2 Hz, 1H), 5.51 (s, 1H), 5.38-5.32 (m, 4H), 5.27 (td, J = 9.3, 5.8 Hz, 3H), 4.99 (dd, J = 17.5, 8.0 Hz, 11H), 4.82 (td, J = 7.5, 4.6 Hz, 2H), 4.63 (dd, J = 8.3, 5.6 Hz, 2H), 4.48- 4.44 (m, 1H), 4.29 (d, J = 10.2 Hz, 1H), 4.13 (q, J = 6.6 Hz, 3H), 3.94 (dd, J = 17.5, 4.9 Hz, 1H), 3.78 (dd, J = 16.0, 6.8 Hz, 1H), 3.20 (qd, J = 15.7, 6.0 Hz, 3H), 2.33 (dd, J = 13.0, 6.0 Hz, 1H), 2.15 (dt, J = 15.2, 7.4 Hz, 2H), 1.88 (t, J = 9.2 Hz, 4H), 1.86 (s, 1H), 1.79 |
| | | (tt, J = 13.2, 6.7 Hz, 2H), 1.74-1.70 |
| | | (m, 2H), 1.70-1.64 (m, 2H), 1.64- |
| | | 1.60 (m, 2H), 1.57 (dd, J = 12.7, 5.1 |
| | | Hz, 1H), 1.56-1.54 (m, 4H), 1.54 (s, |
| | | 4H), 1.09 (d, J = 6.8 Hz, 3H), 0.97 (d, |
| | | J = 6.8 Hz, 3H), 0.76 (d, J = 6.5 Hz, |
| | | 3H), 0.68 (d, J = 6.4 Hz, 3H). |
| | | ¹³C NMR (200 MHz, Pyr) δ 174.95, 173.28, |
| | | 172.92, 172.88, 172.26, 172.13, 171.87, |
| | | 171.26, 171.15, 170.76, 170.49, 160.72, |
| | | 149.38, 149.35, 143.57, 139.86, 134.65, |
| | | 134.11, 133.58, 132.53, 130.34, 129.29, |
| | | 127.49, 127.46, 127.34, 125.26, 125.10, |
| | | 123.40, 121.69, 120.57, 117.68, 76.16, |
| | | 74.60, 73.15, 72.55, 71.94, 70.97, |
| | | 66.83, 65.40, 62.79, 62.31, 61.43, |
| | | 59.15, 54.33, 52.07, 51.07, 48.07, |
| | | 44.55, 44.18, 36.07, 31.15, 29.55, |
| | | 25.60, 25.31, 23.35, 22.43, 21.58, |
| | | 21.28, 18.51, 17.75, 15.23.; |
| | | HRESIMS m/z 1338.5482 [M + Na]⁺ (calcd |
| | | for C₆₂H₆₁N₁₁O₂₁Na, 1338.5506) |
| Chemical Formula 1-2 | 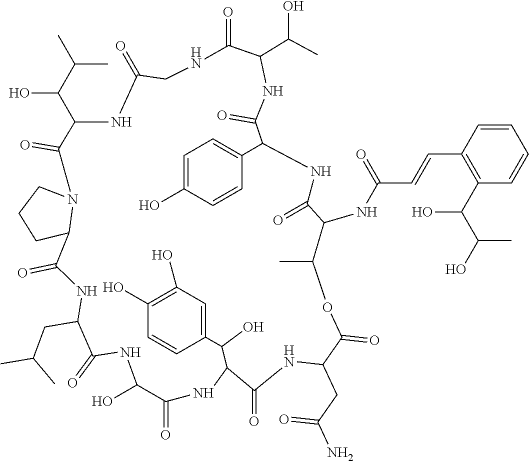 [SMILES: CC(C)CC1NC(=O)C2CCCN2C(=O)C(C(O)C(C)C)NC(=O)CNC(=O)C(C(C)O)NC(=O)C(c2ccc(O)cc2)NC(=O)C(NC(=O)/C=C/c2ccccc2C(O)C(C)O)C(C)OC(=O)C(CC(N)=O)NC(=O)C(C(O)c2ccc(O)c(O)c2)NC(=O)C(O)NC1=O] | ¹H NMR (800 MHz, Pyr) δ 11.11 (d, J = 9.2 Hz, 0H), 9.96 (d, J = 4.1 Hz, 0H), 9.60 (d, J = 7.4 Hz, 0H), 9.45 (d, J = 9.6 Hz, 0H), 9.12 (s, 0H), 9.07 (d, J = 9.8 Hz, 0H), 8.89 (d, J = 15.3 Hz, 0H), 8.84 (d, J = 8.9 Hz, 0H), 8.52 (t, J = 6.1 Hz, 1H), 8.28 (d, J = 3.1 Hz, 0H), 7.96 (s, 0H), 7.81 (t, J = 8.8 Hz, 1H), 7.71 (dd, J = 8.1, 1.9 Hz, 0H), 7.56 (d, J = 2.5 Hz, 0H), 7.45 (d, J = 8.4 Hz, 0H), 7.26 (d, J = 7.7 Hz, 0H), 7.20- 7.17 (m, 1H), 7.10 (d, J = 1.8 Hz, 0H), 6.96 (d, J = 15.3 Hz, 0H), 6.91 (dd, J = 8.5, 2.4 Hz, 0H), 6.70-6.68 (m, 0H), 6.67 (d, J = 7.8 Hz, 0H), 5.98 (dd, J = 13.8, 6.9 Hz, 0H), 5.78 (d, J = 4.3 Hz, 0H), 5.76 (d, J = 7.1 Hz, 0H), 5.73 (d, J = 3.2 Hz, 1H), 5.64 (d, J = 10.2 Hz, 1H), 5.41 (t, J = 9.5 Hz, 1H), 4.98 (dd, J = 17.5, 8.0 Hz, 1H), 4.80 (td, J = 8.0, 4.1 Hz, 0H), 4.64 (dd, J = 8.4, 5.5 Hz, 0H), 4.53-4.50 (m, 0H), 4.27 (d, J = 10.2 Hz, 0H), 4.18 (dd, J = 12.7, 9.5 Hz, 1H), 3.93 (dd, J = 17.5, 4.9 Hz, 0H), 3.77 (dd, J = 16.0, 6.8 Hz, 0H), 3.25 (dd, J = 15.6, 3.8 Hz, 0H), 3.21 (dd, J = 15.5, 8.5 Hz, 0H), 2.53 (dt, J = 13.0, 6.7 Hz, 0H), 2.15 (dt, J = 19.6, 7.6 Hz, 0H), 1.91-1.87 (m, 1H), 1.86 |
| | | (d, J = 6.8 Hz, 1H), 1.80 (ddd, J = |
| | | 18.2, 12.5, 6.6 Hz, 1H), 1.70 (dd, J = |
| | | 14.5, 6.2 Hz, 1H), 1.68 (d, J = 6.1 Hz, |
| | | 1H), 1.68 (t, J = 6.5 Hz, 1H), 1.66 (dd, |
| | | J = 13.0, 6.2 Hz, 0H), 1.62 (td, J = |
| | | 9.8, 6.7 Hz, 1H), 1.48 (d, J = 6.8 Hz, |
| | | 1H), 1.42 (d, J = 6.3 Hz, 1H), 1.17 (d, |
| | | J = 6.8 Hz, 1H), 0.78 (d, J = 6.1 Hz, |
| | | 1H), 0.71 (d, J = 6.0 Hz, 1H). |
| | | ¹³C NMR (200 MHz, Pyr) δ 174.93, 173.34, |
| | | 173.26, 172.89, 172.72, 172.45, 172.05, |
| | | 171.69, 171.14, 171.09, 170.60, 170.40, |
| | | 160.69, 150.58, 150.49, 150.38, 150.24, |
| | | 150.11, 149.95, 149.48, 149.39, 139.47, |
| | | 139.42, 136.59, 136.17, 136.12, 136.01, |
| | | 135.88, 135.76, 135.61, 134.03, 133.81, |
| | | 132.34, 130.23, 129.69, 129.33, 128.73, |
| | | 128.32, 127.75, 125.08, 124.27, 124.16, |
| | | 124.13, 124.10, 123.99, 123.87, 123.75, |
| | | 123.59, 123.47, 121.33, 120.89, 117.66, |
| | | 76.54, 73.00, 72.04, 71.77, 70.91, |
| | | 66.96, 65.64, 64.77, 62.88, 62.19, |
| | | 61.41, 58.97, 54.28, 52.09, 51.11 |
| | | 50.03, 48.07, 44.48, 44.25, 41.43, |
| | | 36.09, 31.15, 29.86, 25.60, 25.35, |
| | | 23.23, 22.57, 22.07, 21.59, 21.02, |
| | | 18.44, 15.51. |
| | | HRESIMS m/z 1356.5100 [M + Na]⁺ calcd |
| | | for C₆₂H₆₃N₁₁O₂₂Na, 1356.5612) |
| Chemical Formula 1-3 | 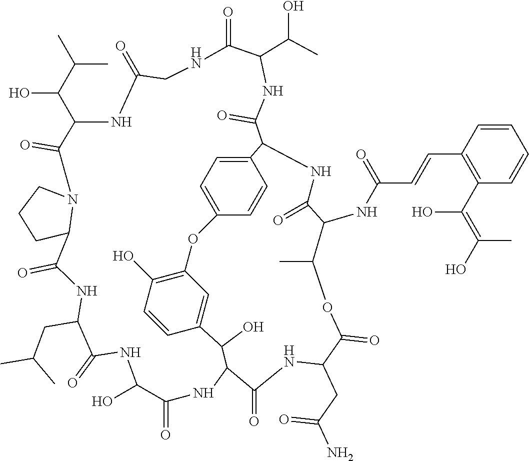 [SMILES: C/C(O)=C(/O)c1ccccc1/C=C/C(=O)NC1C(=O)NC2C(=O)NC(C(C)O)C(=O)NCC(=O)NC(C(O)C(C)C)C(=O)N3CCCC3C(=O)NC(CC(C)C)C(=O)NC(O)C(=O)NC(C(=O)NC(CC(N)=O)C(=O)OC1C)C(O)c1ccc(O)c(c1)Oc1ccc2cc1] | ¹H NMR (800 MHz, Pyr) δ 11.01 (d, J = 9.3 Hz, 1H), 9.98 (d, J = 3.8 Hz, 1H), 9.49 (d, J = 7.4 Hz, 1H), 9.41 (d, J = 9.6 Hz, 1H), 9.15 (d, J = 10.8 Hz, 2H), 8.61-8.57 (m, 1H), 8.53 (s, 1H), 8.38 (d, J = 2.9 Hz, 1H), 8.10 (d, J = 7.8 Hz, 1H), 8.02 (dd, J = 8.5, 2.0 Hz, 1H), 7.95 (s, 1H), 7.72 (dd, J = 8.1, 2.0 Hz, 1H), 7.67 (d, J = 8.4 Hz, 1H), 7.51 (dd, J = 8.1, 2.4 Hz, 1H), 7.32 (d, J = 8.4 Hz, 1H), 7.26 (d, J = 7.8 Hz, 1H), 7.24 (t, J = 6.8 Hz, 2H), 7.12 (dd, J = 8.4, 2.3 Hz, 1H), 7.09 (d, J = 1.8 Hz, 1H), 6.74 (d, J = 9.3 Hz, 2H), 6.58 (t, J = 7.4 Hz, 1H), 6.00 (q, J = 6.9 Hz, 1H), 5.83 (d, J = 4.5 Hz, 1H), 5.76 (d, J = 3.1 Hz, 1H), 5.71 (d, J = 3.9 Hz, 1H), 5.57 (d, J = 10.2 Hz, 1H), 5.51 (s, 1H), 5.38-5.32 (m, 4H), 5.27 (td, J = 9.3, 5.8 Hz, 3H), 4.99 (dd, J = 17.5, 8.0 Hz, 11H), 4.82 (td, J = 7.5, 4.6 Hz, 2H), 4.63 (dd, J = 8.3, 5.6 Hz, 2H), 4.48- 4.44 (m, 1H), 4.29 (d, J = 10.2 Hz, 1H), 4.13 (q, J = 6.6 Hz, 3H), 3.94 (dd, J = 17.5, 4.9 Hz, 1H), 3.78 (dd, J = 16.0, 6.8 Hz, 1H), 3.20 (qd, J = 15.7, 6.0 Hz, 3H), 2.33 (dd, J = 13.0, 6.0 Hz, 1H), 2.15 (dt, J = 15.2, 7.4 Hz, 2H), 1.88 (t, J = 9.2 Hz, 4H), 1.86 (s, 1H), 1.79 |
| | | (tt, J = 13.2, 6.7 Hz, 2H), 1.74-1.70 |
| | | (m, 2H), 1.70-1.64 (m, 2H), 1.64- |
| | | 1.60 (m, 2H), 1.57 (dd, J = 12.7, 5.1 |
| | | Hz, 1H), 1.56-1.54 (m, 4H), 1.54 (s, |
| | | 4H), 1.09 (d, J = 6.8 Hz, 3H), 0.97 (d, |
| | | J = 6.8 Hz, 3H), 0.76 (d, J = 6.5 Hz, |
| | | 3H), 0.68 (d, J = 6.4 Hz, 3H). |
| | | ¹³C NMR (200 MHz, Pyr) δ 174.95, 173.28, |
| | | 172.92, 172.88, 172.26, 172.13, 171.87, |
| | | 171.26, 171.15, 170.76, 170.49, 160.72, |
| | | 156.35, 154.38, 153.35, 155.22, 143.57, |
| | | 139.86, 134.65, 134.11, 133.58, 132.53, |
| | | 130.34, 129.29, 127.49, 127.46, 127.34, |
| | | 125.26, 125.10, 123.40, 121.69, 120.57, |
| | | 117.68, 76.16, 74.60, 73.15, 72.55, |
| | | 71.94, 70.97, 62.79, 62.31, 61.43, |
| | | 59.15, 54.33, 52.07, 51.07, 48.07, |
| | | 44.55, 44.18, 36.07, 31.15, 29.55, |
| | | 25.60, 25.31, 23.35, 22.43, 21.58, |
| | | 21.28, 18.51, 17.75, 15.23.; |
| | | ESIMS m/z 1314.82 [M + H]⁺ |
| Chemical Formula 1-4 | 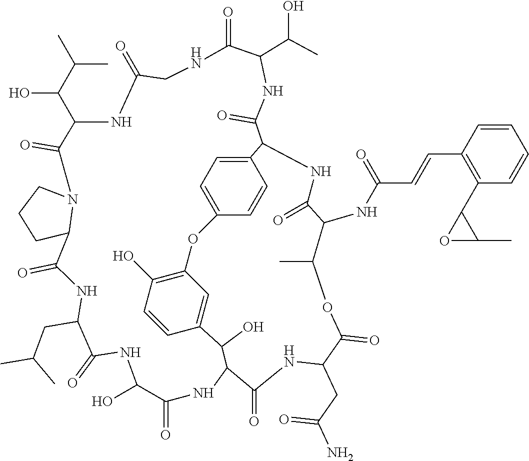 [SMILES: CC(C)CC1NC(=O)C2CCCN2C(=O)C(C(O)C(C)C)NC(=O)CNC(=O)C(C(C)O)NC(=O)C2NC(=O)C(NC(=O)/C=C/c3ccccc3C3OC3C)C(C)OC(=O)C(CC(N)=O)NC(=O)C(NC(=O)C(O)NC1=O)C(O)c1ccc(O)c(c1)Oc1ccc2cc1] | ¹H NMR (800 MHz, Pyr) δ 11.01 (d, J = 9.3 Hz, 1H), 9.98 (d, J = 3.8 Hz, 1H), 9.49 (d, J = 7.4 Hz, 1H), 9.41 (d, J = 9.6 Hz, 1H), 9.15 (d, J = 10.8 Hz, 2H), 8.61-8.57 (m, 1H), 8.53 (s, 1H), 8.38 (d, J = 2.9 Hz, 1H), 8.10 (d, J = 7.8 Hz, 1H), 8.02 (dd, J = 8.5, 2.0 Hz, 1H), 7.95 (s, 1H), 7.72 (dd, J = 8.1, 2.0 Hz, 1H), 7.67 (d, J = 8.4 Hz, 1H), 7.51 (dd, J = 8.1, 2.4 Hz, 1H), 7.32 (d, J = 8.4 Hz, 1H), 7.26 (d, J = 7.8 Hz, 1H), 7.24 (t, J = 6.8 Hz, 2H), 7.12 (dd, J = 8.4, 2.3 Hz, 1H), 7.09 (d, J = 1.8 Hz, 1H), 6.74 (d, J = 9.3 Hz, 2H), 6.58 (t, J = 7.4 Hz, 1H), 6.00 (q, J = 6.9 Hz, 1H), 5.83 (d, J = 4.5 Hz, 1H), 5.76 (d, J = 3.1 Hz, 1H), 5.71 (d, J = 3.9 Hz, 1H), 5.57 (d, J = 10.2 Hz, 1H), 5.51 (s, 1H), 5.38-5.32 (m, 4H), 5.27 (td, J = 9.3, 5.8 Hz, 3H), 4.99 (dd, J = 17.5, 8.0 Hz, 11H), 4.82 (td, J = 7.5, 4.6 Hz, 2H), 4.63 (dd, J = 8.3, 5.6 Hz, 2H), 4.48- 4.44 (m, 1H), 4.29 (d, J = 10.2 Hz, 1H), 4.13 (q, J = 6.6 Hz, 3H), 3.94 (dd, J = 17.5, 4.9 Hz, 1H), 3.78 (dd, J = 16.0, 6.8 Hz, 1H), 3.20 (qd, J = 15.7, 6.0 Hz, 3H), 2.33 (dd, J = 13.0, 6.0 Hz, 1H), 2.15 (dt, J = 15.2, 7.4 Hz, 2H), 1.88 (t, J = 9.2 Hz, 4H), 1.86 (s, 1H), 1.79 |
| | | (tt, J = 13.2, 6.7 Hz, 2H), 1.74-1.70 |
| | | (m, 2H), 1.70-1.64 (m, 2H), 1.64- |
| | | 1.60 (m, 2H), 1.57 (dd, J = 12.7, 5.1 |
| | | Hz, 1H), 1.56-1.54 (m, 4H), 1.54 (s, |
| | | 4H), 1.09 (d, J = 6.8 Hz, 3H), 0.97 (d, |
| | | J = 6.8 Hz, 3H), 0.76 (d, J = 6.5 Hz, |
| | | 3H), 0.68 (d, J = 6.4 Hz, 3H). |
| | | ¹³C NMR (200 MHz, Pyr) δ 174.95, 173.28, |
| | | 172.92, 172.88, 172.26, 172.13, 171.87, |
| | | 171.26, 171.15, 170.76, 170.49, 160.72, |
| | | 149.38, 149.35, 143.57, 139.86, 134.65, |
| | | 134.11, 133.58, 132.53, 130.34, 129.29, |
| | | 127.49, 127.46, 127.34, 125.26, 125.10, |
| | | 123.40, 121.69, 120.57, 117.68, 76.16, |
| | | 74.60, 73.15, 72.55, 71.94, 70.97, |
| | | 66.83, 65.40, 63.79, 63.31, 61.43, |
| | | 59.15, 54.33, 52.07, 51.07, 48.07, |
| | | 44.55, 44.18, 36.07, 31.15, 29.55, |
| | | 25.60, 25.31, 23.35, 22.43, 21.58, |
| | | 21.28, 18.51, 17.75, 15.23.; |
| | | ESIMS m/z 1298.85 [M + H]⁺ |
| Chemical Formula 1-5 | 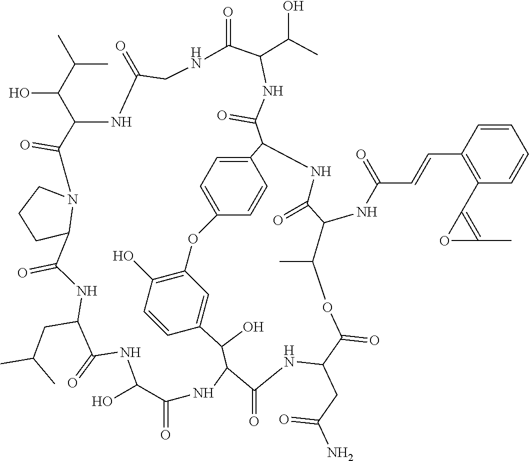 [SMILES: CC1=C(c2ccccc2/C=C/C(=O)NC2C(=O)NC3C(=O)NC(C(C)O)C(=O)NCC(=O)NC(C(O)C(C)C)C(=O)N4CCCC4C(=O)NC(CC(C)C)C(=O)NC(O)C(=O)NC(C(=O)NC(CC(N)=O)C(=O)OC2C)C(O)c2ccc(O)c(c2)Oc2ccc3cc2)O1] | ¹H NMR (800 MHz, Pyr) δ 11.01 (d, J = 9.3 Hz, 1H), 9.98 (d, J = 3.8 Hz, 1H), 9.49 (d, J = 7.4 Hz, 1H), 9.41 (d, J = 9.6 Hz, 1H), 9.15 (d, J = 10.8 Hz, 2H), 8.61-8.57 (m, 1H), 8.53 (s, 1H), 8.38 (d, J = 2.9 Hz, 1H), 8.10 (d, J = 7.8 Hz, 1H), 8.02 (dd, J = 8.5, 2.0 Hz, 1H), 7.95 (s, 1H), 7.72 (dd, J = 8.1, 2.0 Hz, 1H), 7.67 (d, J = 8.4 Hz, 1H), 7.51 (dd, J = 8.1, 2.4 Hz, 1H), 7.32 (d, J = 8.4 Hz, 1H), 7.26 (d, J = 7.8 Hz, 1H), 7.24 (t, J = 6.8 Hz, 2H), 7.12 (dd, J = 8.4, 2.3 Hz, 1H), 7.09 (d, J = 1.8 Hz, 1H), 6.74 (d, J = 9.3 Hz, 2H), 6.58 (t, J = 7.4 Hz, 1H), 6.00 (q, J = 6.9 Hz, 1H), 5.83 (d, J = 4.5 Hz, 1H), 5.76 (d, J = 3.1 Hz, 1H), 5.71 (d, J = 3.9 Hz, 1H), 5.57 (d, J = 10.2 Hz, 1H), 5.51 (s, 1H), 5.38-5.32 (m, 4H), 5.27 (td, J = 9.3, 5.8 Hz, 3H), 4.99 (dd, J = 17.5, 8.0 Hz, 11H), 4.82 (td, J = 7.5, 4.6 Hz, 2H), 4.63 (dd, J = 8.3, 5.6 Hz, 2H), 4.48- 4.44 (m, 1H), 4.29 (d, J = 10.2 Hz, 1H), 4.13 (q, J = 6.6 Hz, 3H), 3.94 (dd, J = 17.5, 4.9 Hz, 1H), 3.78 (dd, J = 16.0, 6.8 Hz, 1H), 3.20 (qd, J = 15.7, 6.0 Hz, 3H), 2.33 (dd, J = 13.0, 6.0 Hz, 1H), 2.15 (dt, J = 15.2, 7.4 Hz, 2H), 1.88 (t, J = 9.2 Hz, 4H), 1.86 (s, 1H), 1.79 |
| | | (tt, J = 13.2, 6.7 Hz, 2H), 1.74-1.70 |
| | | (m, 2H), 1.70-1.64 (m, 2H), 1.64- |
| | | 1.60 (m, 2H), 1.57 (dd, J = 12.7, 5.1 |
| | | Hz, 1H), 1.56-1.54 (m, 4H), 1.54 (s, |
| | | 4H), 1.09 (d, J = 6.8 Hz, 3H), 0.97 (d, |
| | | J = 6.8 Hz, 3H), 0.76 (d, J = 6.5 Hz, |
| | | 3H), 0.68 (d, J = 6.4 Hz, 3H). |
| | | ¹³C NMR (200 MHz, Pyr) δ 174.95, 173.28, |
| | | 172.92, 172.88, 172.26, 172.13, 171.87, |
| | | 171.26, 171.15, 170.26, 170.49, 160.72, |
| | | 156.35, 154.38, 149.38, 149.35, 143.57, |
| | | 139.86, 134.65, 134.11, 133.58, 132.53, |
| | | 130.34, 129.29, 127.49, 127.46, 127.34, |
| | | 125.26, 125.10, 123.40, 121.69, 120.57, |
| | | 117.68, 76.16, 74.60, 73.15, 72.55, |
| | | 71.94, 70.97, 66.83, 65.40, 63.79, |
| | | 59.15, 54.33, 52.07, 51.07, 48.07, |
| | | 44.55, 44.18, 36.07, 31.15, 29.55, |
| | | 25.60, 25.31, 23.35, 22.43, 21.58, |
| | | 21.28, 18.51, 17.75, 15.23.; |
| | | ESIMS m/z 1296.85 [M + H]⁺ |
| Chemical Formula 1-6 | 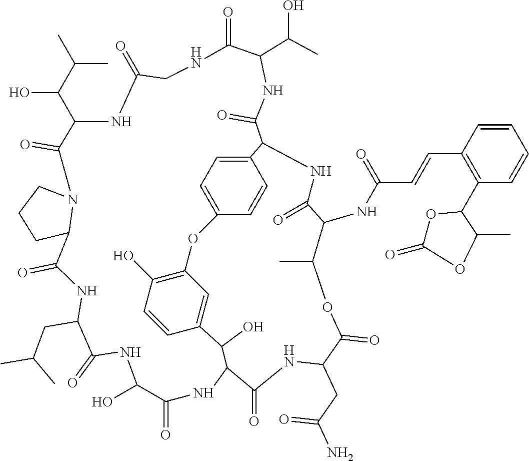 [SMILES: CC(C)CC1NC(=O)C2CCCN2C(=O)C(C(O)C(C)C)NC(=O)CNC(=O)C(C(C)O)NC(=O)C2NC(=O)C(NC(=O)/C=C/c3ccccc3C3OC(=O)OC3C)C(C)OC(=O)C(CC(N)=O)NC(=O)C(NC(=O)C(O)NC1=O)C(O)c1ccc(O)c(c1)Oc1ccc2cc1] | ¹H NMR (800 MHz, Pyr) δ 10.81 (d, J = 9.2 Hz, 1H), 9.96 (d, J = 3.8 Hz, 1H), 9.60 (d, J = 7.0 Hz, 1H), 9.33 (d, J = 9.5 Hz, 1H), 9.21 (s, 1H), 9.04 (d, J = 10.0 Hz, 1H), 8.72 (s, 3H), 8.64-8.59 (m, 1H), 8.59-8.53 (m, 2H), 8.34 (s, 1H), 7.97 (s, 1H), 7.76 (d, J = 8.3 Hz, 2H), 7.70 (d, J = 8.0 Hz, 1H), 7.54 (d, J = 8.2 Hz, 2H), 7.49 (d, J = 7.9 Hz, 1H), 7.44 (d, J = 7.8 Hz, 1H), 7.40 (d, J = 8.3 Hz, 1H), 7.33-7.27 (m, 2H), 7.29 (d, J = 15.4 Hz, 1H), 7.12 (s, 1H), 7.04 (s, 1H), 6.81 (t, J = 7.5 Hz, 1H), 6.76 (d, J = 9.8 Hz, 1H), 6.74 (dd, J = 16.8, 9.5 Hz, 2H), 6.73 (d, J = 9.2 Hz, 1H), 6.26 (d, J = 7.6 Hz, 1H), 6.00 (dd, J = 13.4, 6.6 Hz, 2H), 6.01-5.98 (m, 1H), 5.78 (d, J = 3.0 Hz, 1H), 5.74 (d, J = 4.1 Hz, 1H), 5.63 (d, J = 10.1 Hz, 1H), 5.52 (s, 1H), 5.45-5.41 (m, 1H), 5.45-5.41 (m, 1H), 5.35 (d, J = 9.9 Hz, 1H), 5.24 (d, J = 5.7 Hz, 1H), 5.23 (s, 2H), 5.20 (dd, J = 14.9, 9.2 Hz, 2H), 4.92 (d, J = 7.5 Hz, 2H), 4.93- 4.88 (m, 6H), 4.90 (s, 2H), 4.87-4.83 (m, 3H), 4.62-4.58 (m, 1H), 4.29 (d, J = 10.2 Hz, 1H), 4.13 (dd, J = 15.9, 7.0 Hz, 2H), 4.07 (d, J = 9.7 Hz, 1H), 3.95 (dd, J = 17.4, 4.9 Hz, 1H), 3.71 (dd, J = |
| | | 15.7, 6.7 Hz, 1H), 3.63-3.57 (m, |
| | | 1H), 3.23 (dd, J = 23.7, 15.8 Hz, 2H), |
| | | 2.40-2.35 (m, 1H), 2.16 (dd, J = 12.4, |
| | | 7.4 Hz, 2H), 2.16 (dd, J = 12.4, 7.4 Hz, |
| | | 2H), 1.87 (d, J = 6.9 Hz, 3H), 1.82- |
| | | 1.77 (m, 2H), 1.82-1.77 (m, 2H), 1.73- |
| | | 1.70 (m, 2H), 1.67 (d, J = 6.2 Hz, |
| | | 3H), 1.66-1.63 (m, 2H), 1.63-1.58 |
| | | (m, 3H), 1.56 (d, J = 6.4 Hz, 3H), 1.15 |
| | | (d, J = 6.7 Hz, 4H), 1.11 (d, J = 6.8 |
| | | Hz, 3H), 0.79 (d, J = 6.4 Hz, 4H), 0.75 |
| | | (d, J = 6.3 Hz, 3H). |
| | | ¹³C NMR (200 MHz, Pyr) δ 174.96, 173.29, |
| | | 173.21, 173.10, 172.82, 172.39, 171.97, |
| | | 171.70, 171.17, 171.08, 170.72, 170.28, |
| | | 160.72, 155.19, 150.58, 150.49, 150.38, |
| | | 150.24, 150.11, 149.96, 149.54, 149.41, |
| | | 137.93, 136.28, 136.17, 136.11, 136.00, |
| | | 135.88, 135.76, 135.61, 134.23, 133.78, |
| | | 133.45, 132.46, 130.38, 130.29, 130.24, |
| | | 129.00, 127.93, 127.23, 125.18, 124.16, |
| | | 124.10, 123.99, 123.87, 123.75, 123.59, |
| | | 121.64, 120.70, 117.81, 81.32, 80.56, |
| | | 76.52, 73.27, 71.77, 70.74, 67.10, |
| | | 65.63, 63.13, 62.12, 61.46, 59.33, |
| | | 53.85, 52.13, 51.13, 48.20, 44.64, |
| | | 44.35, 36.12, 31.20, 29.71, 25.60, |
| | | 25.34, 23.40, 22.47, 21.55, 21.36, |
| | | 18.62, 18.42, 15.40.; |
| | | HRESIMS m/z 1364.5276 [M + Na]⁺ (calcd |
| | | for C₆₃H₇₉N₁₁O₂₂Na, 1364.5299) |

### Experimental Example 1

### Analysis of Wnt Activities of Chemical Formulas 1-1 to 1-6

In order to measure the level of Wnt activities of Chemical Formulas 1-1 to 1-6, TopFLASH luciferase assay was performed on the HEK293T cell lines (FIG. 1).

In order to measure the level of Wnt activities of Chemical Formulas 1-1 to 1-6, TopFLASH luciferase assay was performed on the HEK293T cell lines. After generating 7TFC virus plasmid (Addgene, Plasmid #24307), 293T cells were inserted thereinto. Cells with mCherry signals were selected under a fluorescence microscope (ZEISS, Observer Z1). RPE cells were transfected with the M50 Super 8× TOPFlash plasmid (Addgene, Plasmid #12456) using Lipofectamine 3000 transfection reagent (Thermo Fisher Scientific, L3000-015) according to the manufacturer's protocol. After incubation for 36 hours, the transfected cells were replaced with fresh media and treated with Wintamide in the presence or absence of Wnt3a-CM (10%) for 24 hours at 37° C. in a 5% CO₂ incubator.

In 96 well plates (1×10⁴ cells/well), 293T-7TFC and M50 Super 8×-transfected RPE cells were cultured. After overnight incubation, cells were treated with the indicated concentrations of Chemical Formulas 1-1, 1-2, 1-3, 1-4, 1-5 and 1-6 and Wnt3a-CM (10%), followed by incubation for 24 hours. Cells were treated with One-Glo™ used as a detector in the TOPFlash luciferase assay according to the manufacturer's instructions. Luciferase intensity was measured using a photometer (Perkin Elmer, victor™ X2).

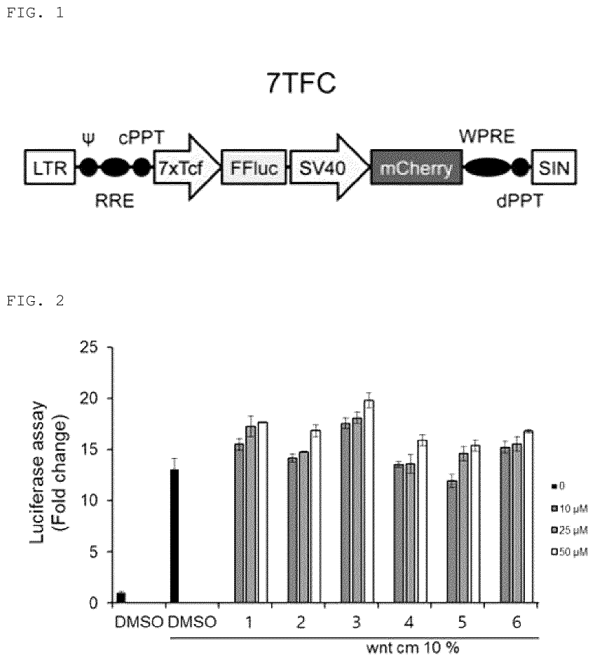

As could be confirmed in FIG. 2, the luciferase values were found to increase significantly as the concentration increased during the treatment of Chemical Formulas 1-1 to 1-6. Thus, the compounds of the present invention were found to greatly enhance Wnt activity.

### Experimental Example 2

### Analysis of Toxicity and Inhibition of Adipogenesis in Various Cell lines for Chemical Formula 1-1

### Experimental Example 2-1

### Various Cytotoxicity Assays

To measure the cytotoxicity of the compound represented by Chemical Formula 1 (Chemical Formula 1-1), toxicity was measured in mouse preadipocyte 3T3-L1 cell line, human malignant melanoma A375 cell line, mouse malignant melanoma B16F10 cell line, human oral cancer MDA-MB-435 cell line, mouse breast cancer 4T1 cell line, human breast cancer MCF7 cell line, human brain cancer U87MG cell line, human glioblastoma U373MG cell line, human cervical cancer HeLa cell line, mouse embryonic fibroblast (MEF) cell line, human embryonic kidney HEK293T cell line (Korea Cell Line Bank). First, 1×10⁴ cells were divided into 200 μl each in a 96-well plate using Dulbecco's Modified Eagle's Medium (DMEM) supplemented with 10% fetal bovine serum (FBS), 50 mg/ml streptomycin, and 50 U/ml penicillin, while maintaining various cells at 37r in an atmosphere of 5% CO₂. The cells were treated with the compound of Chemical Formula 1-1 of the present invention at a maximum concentration of 100 μM and cultured for 48 hours under the same culture conditions. After the incubation, the culture media were removed and the cytotoxicity test results were confirmed (Table 2 and FIG. 3 below).

As a result, as shown in Table 2 below, the compound of Chemical Formula 1-1 showed no cytotoxicity even in various cell lines.

TABLE 2

| Cell line | IC₅₀ (μM) |
|---|---|
| mouse preadipocyte 3T3-L1 cell line | >100 |
| human malignant melanoma A375 cell line | >100 |
| mouse malignant melanoma B16F10 cell line | >100 |
| human oral cancer MDA-MB-435 cell line | >100 |
| mouse breast cancer 4T1 cell line | >100 |
| human breast cancer MCF7 cell line | >100 |
| human brain cancer U87MG cell line | >100 |
| human glioblastoma U373MG cell line | 89.5 |
| human cervical cancer HeLa cell line | >100 |
| mouse embryonic fibroblast (MEF) cell line | >100 |
| human embryonic kidney HEK293T cell line | >100 |

Next, in order to confirm the cytotoxicity in the Chemical Formula 1-1 and wnt3 culture, toxicity in 3T3-L1 cells was measured on the compound of Chemical Formula 1-1 of the present invention at a concentration of 2.5 to 10 μM in L-conditional media or Wnt3a-containing culture media (2.5%). As a result, as shown in FIG. 3, cytotoxicity was not observed even in the Wnt3a-containing culture media.

### Experimental Example 2-2

### Wnt3a Activity Assay

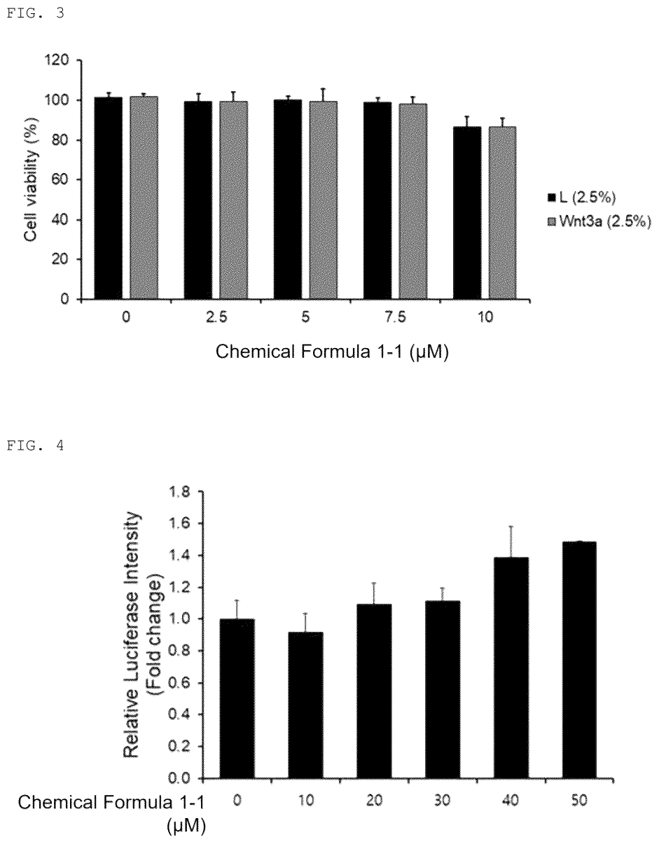

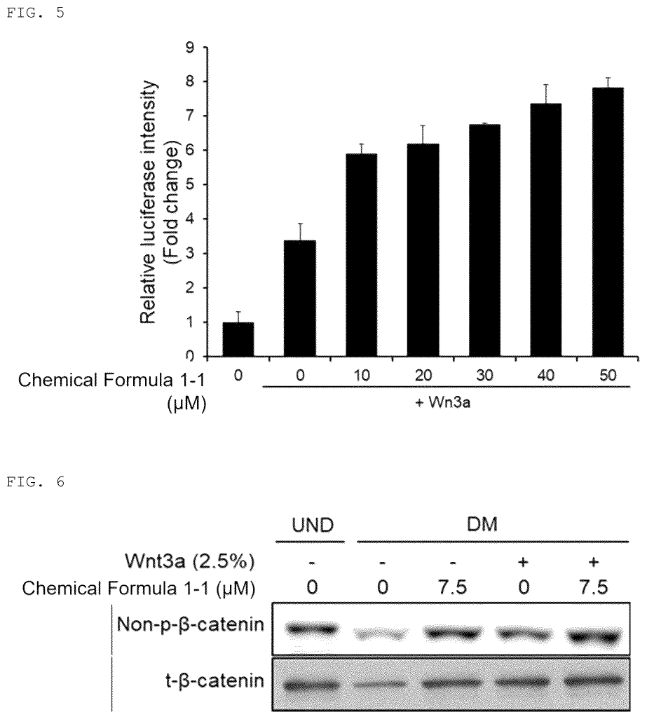

In order to measure the level of Wnt activity of the compound of Chemical Formula 1-1, TopFLASH luciferase assay was performed on the HEK293T cell line. The TopFLASH luciferase assay was performed by creating a HEK293T-TopFLASH stable cell line with a viral vector made by combining Luciferase (reporter gene) with the TCF7 promoter. HEK293T-TopFLASH cells were cultured in a 96-well plate (1×10⁴ cells/well), and the next day, the cells were treated with 1-50 μM of the compound of Chemical Formula 1-1 and confirmed by One-Glo™ (modified luciferin, Pormega, USA) after 24 hours. As shown in TopFLASH assay results of FIGS. 4 and 5, as a result of treatment with the compound of Chemical Formula 1-1 alone and simultaneous treatment with the compound of Chemical Formula 1-1 and Wnt3a-conditional media (2.5%), it was confirmed that as the luciferase values greatly increased, Wnt activity greatly increased when treated with Chemical Formula 1-1.

### Experimental Example 2-3

### Analysis of Changes in Wnt3a Signaling Protein

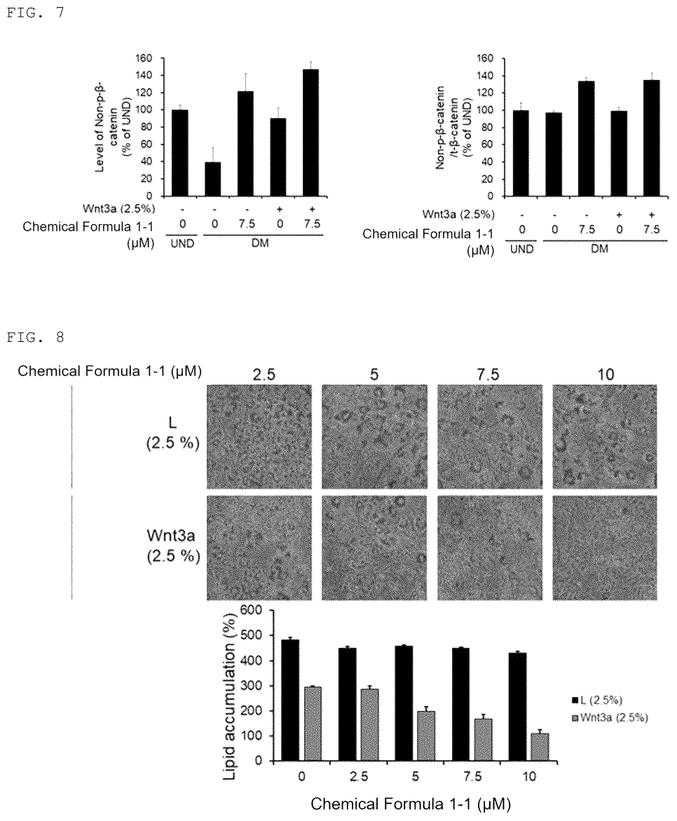

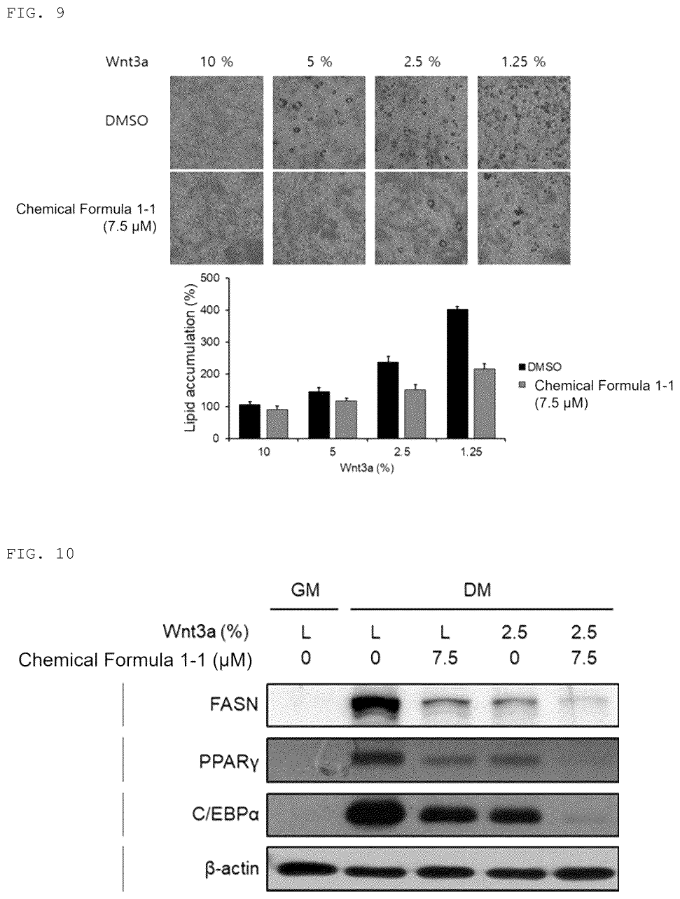

To confirm the Wnt3a signaling of Chemical Formula 1-1, the protein level of β, a Wnt3a signaling protein, was measured in the 3T3-L1 cell line. The 3T3-L1 cells were cultured in a 6-well plate (2×10⁵ cell/well), and the next day, the cells were replaced with normal culture media (Undifferentiated media; UND) and differentiation induction media (Differentiation media; DM), respectively, followed by treatment with the compound of Chemical Formula 1-1 at 7.5 μM for 48 hours, and then the cells were replaced with new normal culture media (Maintaining media; MM). Here, the compound of Chemical Formula 1-1 at a concentration of 7.5 μM was cultured for 72 hours, followed by western blotting. In FIGS. 6 and 7, it was confirmed that the protein level of β increased in the treatment of the compound of Chemical Formula 1-1 alone and the treatment of Chemical Formula 1-1 and Wnt3a together.

### Experimental Example 2-4

### Adipogenesis Inhibitory Effect Test (1)

The adipogenesis inhibitory activity of Chemical Formula 1 according to the present invention was measured through Oil Red O staining in mouse 3T3-L1 cell line. First, 3T3-L1 cells were placed in a 48-well plate (1×10⁴ cell/well) and cultured for 24 hours in a 37° C. CO₂ incubator. The next day, the cells were replaced with the normal culture media (Undifferentiated media; UND) and the differentiation induction medium (Differentiation media; DM), respectively, followed by treatment with 2.5-10 μM of Chemical Formula 1-1 for 48 hours. Then, the cells were replaced with the normal culture media (Maintainment media; MM), and cultured with 2.5-10 μM of Chemical Formula 1-1 for 72 hours, and then treated with Oil Red O.

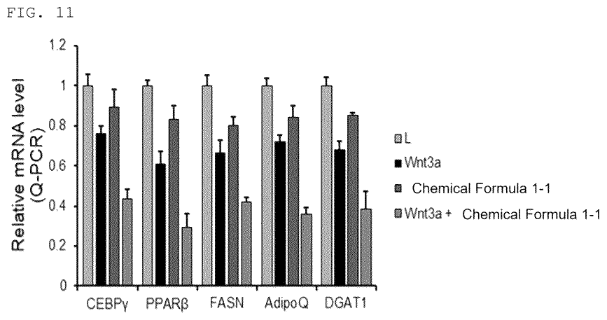

As a result, as shown in FIG. 8, it was confirmed that the compound of Chemical Formula 1-1 exhibited the adipogenesis inhibitory activity in a concentration-dependent manner.

In addition, the same experiment was conducted with the compound of Chemical Formula 1-1 at a fixed concentration of 7.5 μM and Wnt3a-conditional media at 10-2.5%.

As shown in FIG. 9, it was confirmed that the adipogenesis inhibitory activity was excellent even when treated with the compound of Chemical Formula 1-1 even under the Wnt3a concentration condition.

### Experimental Example 2-5

### Adipogenesis Inhibitory Effect Test (2)

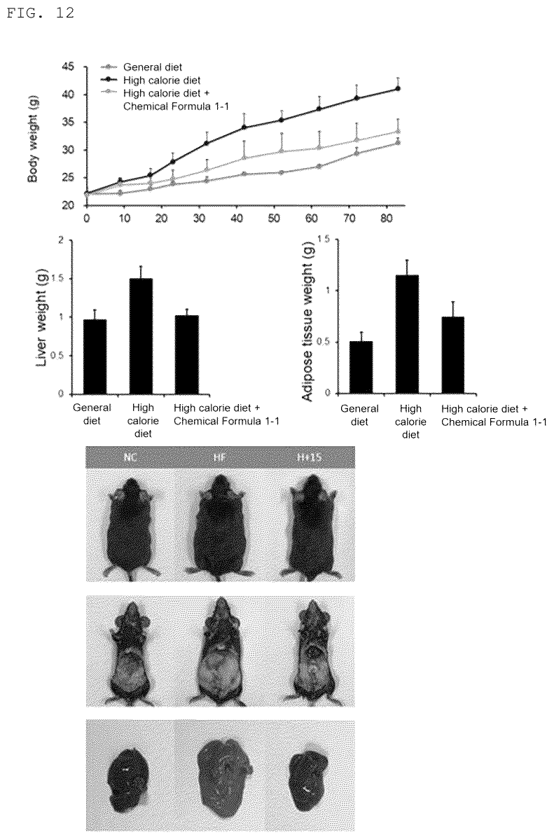

Additionally, in order to verify the adipogenesis inhibitory ability of the Chemical Formula 1 according to the present invention, the level of adipocyte differentiation-associated proteins in the 3T3-L1 cell line was measured. The 3T3-L1 cells were cultured in a 6-well plate (2×10⁵ cell/well), and the next day, the cells were replaced with normal culture media (Undifferentiated media; UND) and differentiation induction media (Differentiation media; DM), respectively, followed by treatment with the compound of Chemical Formula 1-1 at 7.5 μM for 48 hours, and then the cells were replaced with new normal culture media (Maintaining media; MM). When replacement with normal culture media, the cells were treated with 7.5 μM of Chemical Formula 1-1, and cultured for 72 hours more, followed by Western blotting. FIG. 10 shows the results of lower protein levels in differentiated adipocytes in the treatment with the compound of Chemical Formula 1-1 alone and simultaneous treatment with the compound of Chemical Formula 1-1 and Wnt3a-conditional media (2.5%), and it was confirmed that the level of adipogenesis induction-associated proteins was reduced when treated with the compound of Chemical Formula 1 according to the present invention.

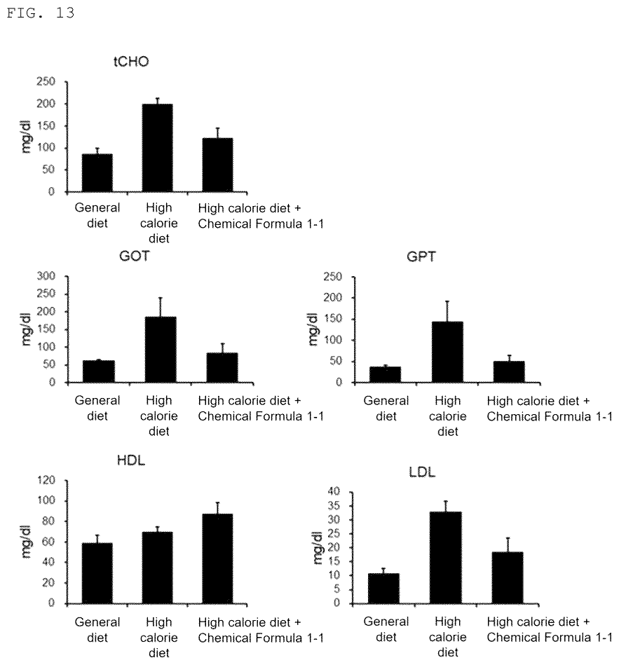

In addition, real-time polymerase chain reaction (Q-PCR) was used to confirm the expression of adipogenesis-associated genes. As a result, it was confirmed that the amount of adipogenesis induction-associated mRNA was reduced when treated with the compound of Chemical Formula 1 according to the present invention (FIG. 11).

### Experimental Example 2-6

### Verification in Animal Models

C57BL/C6 mice were used to test the anti-obesity effect in a mouse model with respect to the compound of Chemical Formula 1 of the present invention, which is an adipogenesis-inhibiting compound. For effective evaluation of the anti-obesity model, the control group (general diet), the control group (high calorie diet), and an anti-obesity control group (high calorie diet+Chemical Formula 1 (15 mg/kg)) were compared and evaluated (FIG. 12). The mice injected with the high-calorie diet rather than the general diet gained 131% in body weight, but the mice treated with the high-calorie diet and Chemical Formula 1 (15 mg/kg) together lost 115% in body weight. In addition, there was a significant decrease in the measurement of liver weight and abdominal fat weight. In summary of these results, the Chemical Formula 1 was confirmed to have excellent anti-obesity activity in a mouse animal model (FIG. 12).

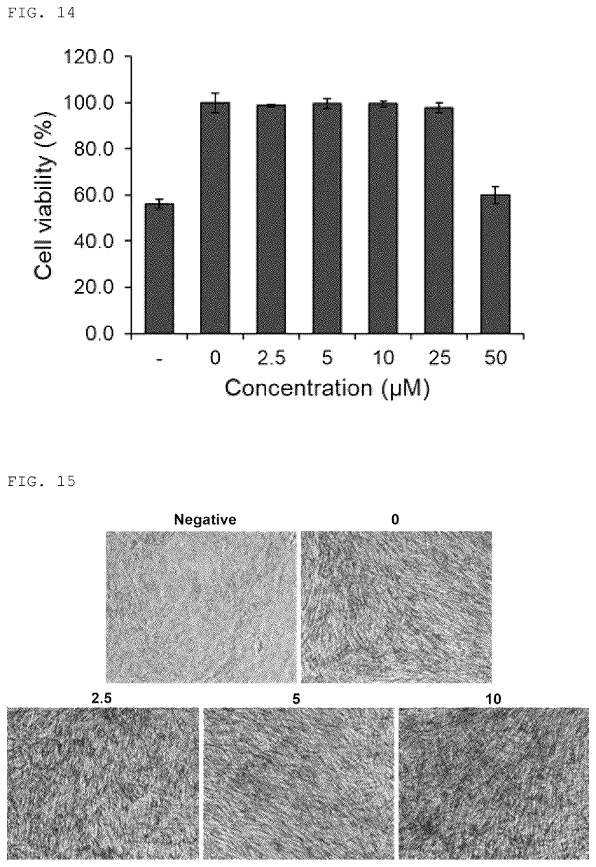

Then, after the experiment was completed, the animal blood of each group was collected and centrifuged to separate plasma, and then representative obesity markers in the blood such as total cholesterol (tCHO), high density lipoprotein (HDL), and low density lipoprotein (LDL), and liver damage markers such as glutamic oxaloacetic transaminase (GOT) and glutamic pyruvic acid transaminase (GPT) were analyzed with a blood chemistry analyzer. As a result, it was confirmed that when compared to the control group with a high calorie intake, tCHO, GOT, GPT, and LDL among the obesity and liver damage markers were significantly reduced, whereas HDL increased compared to the control group with a high calorie intake (FIG. 13).

It was found from these results that the compound represented by Chemical Formula 1 according to the present invention not only inhibits obesity, but also helps improve liver damage and fatty liver formation caused by obesity.

### Statistical Analysis

All values are presented as mean value ±standard error (standard error of the mean: SEM).

### Experimental Example 3

### Analysis of Osteogenesis Properties of Bicyclic Compound According to Chemical Formula 1 of the Present Invention

### Experimental Example 3-1

### Cytotoxicity Assay

In order to measure the cytotoxicity of the compound represented by Chemical Formula 1 (Chemical Formula 1-1), toxicity was measured in the MC3T3-E1 cell line (Korea Cell Line Bank), which is a pre-osteoblast related to bone formation.

Specifically, a cytotoxicity test was performed to determine the in vivo safety of Chemical Formula 1-1 above. MC3T3-E1 cells, which are mouse osteoblasts, were inoculated into a 96-well plate at a concentration of 5×10³ cell/well, and incubated overnight in growth media containing alpha-minimum essential media (alpha-MEM, JBI #008-53) supplemented with 10% fetal bovine serum (FBS, Gibco #16000, USA) and 1× antibiotics (Gibco #15240062, USA). Then, the cells were exchanged with media added with compounds at the indicated concentrations (0, 2.5, 5, 10, 25 and 50 mM) on Day 3, and treated for 7 days. After incubation, the media were removed, 90 μl of the media and 10 μl of WTS solution were added, and after 1 hour, the absorbance was measured at 450 nm using a microplate reader. Results thereof are shown in FIG. 14.

As could be confirmed in FIG. 14, it was confirmed that the Chemical Formula 1-1 prepared in the present invention did not exhibit cytotoxicity in a concentration range of 50 μM or less.

### Experimental Example 3-2

### Analysis of Osteogenesis Effect Through ALP Activity Analysis (1)

In order to analyze the effect of the compound of Chemical Formula 1 according to the present invention on ALP activity, the morphology of MC3T3-E1 cells according to the treatment with the compound of Chemical Formula 1 according to the present invention was confirmed by alkaline phosphatase (ALP) staining of the MC3T3-E1 cell line.

In the present Example, the effect of Chemical Formula 1-1 on the activity of alkaline phosphatase (ALP), which is one of the representative markers of osteoblast differentiation, was measured. MC3T3-E1 cells, which are mouse osteoblasts, were inoculated into a 96-well plate at a concentration of 5×10³ cell/well, and incubated overnight in growth media containing alpha-minimum essential media (alpha-MEM, JBI #008-53) supplemented with 10% fetal bovine serum (FBS, Gibco #16000, USA) and 1× antibiotics (Gibco #15240062, USA). As a negative control, an alpha minimum essential medium containing 10% FBS was used. As positive controls, 100 μg/ml of ALP-activating ascorbic acid and 10 mM of β-glycerophosphate were mixed, and the positive controls were exchanged with media added with 5, 10, and 20 νM of Chemical Formula 1-1 on Day 3, and treated for 7 days. After 7 days, the media were discarded, and the cells were washed with PBS, and fixed with 10% formalin for 1 minute. Then, after washing twice with PBS, the cells were stained with a BCIP/NBT solution (Sigma). When the cells were properly stained, the solution was discarded, and the cells were washed with PBS and dried. The experimental results are shown in FIG. 15.

Referring to FIG. 15, as to Chemical Formula 1-1, alkaline phosphatase staining (ALP) positive cells were strongly stained in a concentration-dependent manner. It was confirmed from these results that the compound represented by Chemical Formula 1 according to the present invention was excellent in promoting osteogenesis.

### Experimental Example 3-3

### Analysis of Osteogenesis Effect Through ALP Activity Analysis (2)

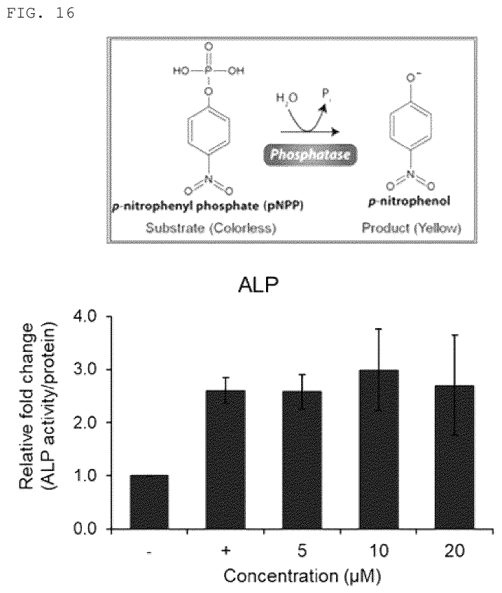

In order to analyze the effect of the compound of Chemical Formula 1 according to the present invention on ALP activity, MC3T3-E1 cells, which are mouse osteoblasts, were inoculated into a 96-well plate at a concentration of 5×10³ cell/well, and incubated overnight in growth media containing alpha-minimum essential media (alpha-MEM, JBI #008-53) supplemented with 10% fetal bovine serum (FBS, Gibco #16000, USA) and 1× antibiotics (Gibco #15240062, USA). As a negative control, an alpha minimum essential medium containing 10% FBS was used. As a positive control, 100 μg/ml of ALP-activating ascorbic acid and 10 mM of β-glycerophosphate were mixed, and the positive control was exchanged with media added with 5, 10, and 20 νM of Chemical Formula 1-1 (6-J) on Day 3, and treated for 7 days. After 7 days, the media were removed and the cells were washed with PBS, and the cultured MC3T3-E1 cells were lysed with 0.1% TritonX-100 (in PBS) solution and centrifuged to obtain only the supernatant. A predetermined amount of supernatant and ALP were added and reacted for 30 minutes, and then the absorbance was confirmed at 405 nm. The experimental results are shown in FIG. 16.

As could be seen in FIG. 16, it was confirmed that the compound of Chemical Formula 1 according to the present invention increased the ALP activity of MC3T3-E1 cells, thereby increasing the differentiation of osteoblasts, indicating that the compound of Chemical Formula 1 according to the present invention is effective in promoting bone growth.

### Experimental Example 3-4

### Analysis of Expression Level Increasing Effect of Osteogenic Differentiation Promoting Gene

In order to confirm the effect of the compound of Chemical Formula 1 according to the present invention on promoting osteogenic differentiation-related genes, the expression levels of genes promoting osteogenesis were confirmed.

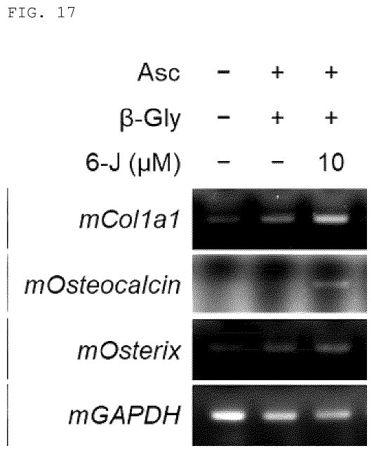

To confirm genetic changes during the differentiation process, total RNA was isolated on Day 7 of differentiation using Trizol reagent (Invitrogen, CA, USA) according to the manufacturer's instructions. The cultured cells were washed twice with cold PBS and lysed with 1 ml of Trizol reagent. To the resulting product, 200 μl of chloroform was added and mixed, followed by centrifugation at 4° C. and 12000 rpm for 20 minutes to separate the supernatant. The same amount of isopropanol was added to the separated supernatant, mixed, and centrifuged again at 4° C. and 12000 rpm. The supernatant was removed, and the remaining pellet was washed three times with 70% ethanol to separate RNA. For RT-PCR, the same amount of total RNA (5 μl) was put into the RT-PCR amplification kit and reacted at 45° C. for 60 minutes to prepare complementary DNA (cDNA). The cDNA was amplified by RT-PCR using the primers shown in Table 3 below. RT-PCR was performed on the cDNA by using AccuPower®RT/PCR PreMix (Bioneer, Korea). Briefly, 2 μl each of 75 mM rATP, rUTP, rCTP, rGTP, enzyme mixture, and 10× reaction buffer was added to 8 μl of the cDNA solution prepared above, followed by amplification according to the manufacturer's instructions, and the quantity and quality of the amplified mRNA was evaluated using nanodrops and 1% agarose gel electrophoresis. Results thereof are shown in FIG. 17.

TABLE 3

| | | Anneal- |
|---|---|---|
| | | ling |
| | | Temper- |
| | | ature |
| Primer | Sequence | (° C.) |
| mColla1 | Forward | 60 |
| | 5′-CGA AGG CAA CAG | |
| | TCG CTT CA-3′ | |
| | (SEQ ID NO: 2) | |
| | Reverse | |
| | 5′-CC CAA GTT CCG | |
| | GTG TGA-3′ | |
| | (SEQ ID NO: 3) | |
| mOsteocalcin | Forward | 60 |
| | 5′-CCT GAG TCT GAC | |
| | AAA GCC TTC A-3′ | |
| | (SEQ ID NO: 4) | |
| | Reverse | |
| | 5′-GCC GGA GTC TGT | |
| | TCA CTA CCT T-3′ | |
| | (SEQ ID NO: 5) | |
| mOsterix | Forward | 58 |
| | 5′-AGC GAC CAC TTG | |
| | AGC AAA CAT-3′ | |
| | (SEQ ID NO: 6) | |
| | Reverse | |
| | 5′-GCG GCT GAT TGG | |
| | CTT CTT CT-3′ | |
| | (SEQ ID NO: 7) | |
| Mgapdh | Forward | 60 |
| | 5′-CAT GGC CTC | |
| | CAAA GGA GTA AGA-3′ | |
| | (SEQ ID NO: 8) | |
| | Reverse | |
| | 5′-GAG GGA GAT GCT | |
| | CAG TGT TGG-3′ | |
| | (SEQ ID NO: 9) | |

As could be seen in FIG. 17, the treatment with the compound of Formula 1-1 of the present invention was found to increase the mRNA expression level of osteogenic differentiation-related genes (mCol1a1, mOsteocalcin, mOsterix).

Therefore, it was found that the compound represented by Chemical Formula 1-1 according to the present invention had an excellent ability to promote osteogenic differentiation.

As described above, the present invention has been described in an exemplary manner, as an example, and various modifications may be made by those skilled in the art to which the present invention pertains without departing from the essential characteristics of the present invention. Therefore, exemplary embodiments disclosed in the present specification are intended to explain the present invention rather than limiting it, and the spirit and scope of the present invention are not limited by these exemplary embodiments. The protection scope of the present invention should be construed by the following claims, and all techniques within the equivalent range should be construed as being included in the scope of the present invention.

1. A compound represented by the following Chemical Formula 1, an optical isomer thereof, or a pharmaceutically acceptable salt thereof: 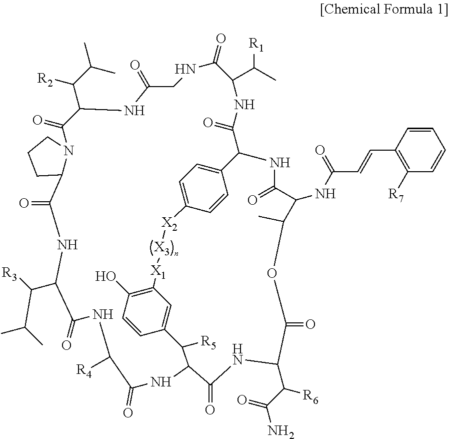
in Chemical Formula 1 above,
R₁, R₂, R₃, R₄, R₅ and R₆ are the same as or different from each other, and are each independently a hydrogen atom or hydroxyl (—OH);
R₇ is a substituted or unsubstituted C₁₋₆ alkyl, a substituted or unsubstituted C₂₋₆ alkene, a substituted or unsubstituted C₃₋₈ heterocycloalkyl containing at least one oxygen atom (O) as a heteroatom, or a substituted or unsubstituted C₃₋₈ heterocycloalkene containing at least one oxygen atom (O) as a heteroatom (wherein at least one H of the substituted C₁₋₆ alkyl, the substituted C₂₋₆ alkene, the substituted C₃₋₈ heterocycloalkyl, or the substituted C₃₋₈ heterocycloalkene may each independently be substituted with hydroxyl (—OH), a halogen atom, C₁₋₆ alkyl or oxo (═O));
n is an integer of either 0 or 1;
10 when n is 0, X₁ and X₂ are the same as or different from each other, and are each independently a hydrogen atom or hydroxyl (—OH); and
when n is 1, X₁ and X₂ are each independently a single bond, and X₃ is —O—.

2. The compound of claim 1, wherein the compound represented by Chemical Formula 1 is a compound represented by the following Chemical Formula 2, an optical isomer thereof, or a pharmaceutically acceptable salt thereof: 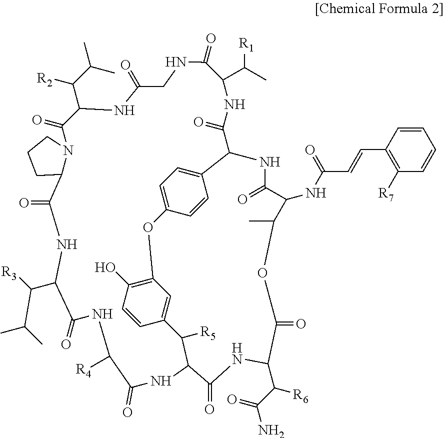
in Chemical Formula 2 above,
R₁, R₂, R₃, R₄, R₅ and R₆ are the same as or different from each other, and are each independently a hydrogen atom or hydroxyl (—OH); and
R₇ is a substituted or unsubstituted C₁₋₆ alkyl, a substituted or unsubstituted C₂₋₆ alkene, a substituted or unsubstituted C₃₋₈ heterocycloalkyl containing at least one oxygen atom (O) as a heteroatom, or a substituted or unsubstituted C₃₋₈ heterocycloalkene containing at least one oxygen atom (O) as a heteroatom (wherein at least one H of the substituted C₁₋₆ alkyl, the substituted C₂₋₆ alkene, the substituted C₃₋₈ heterocycloalkyl, or the substituted C₃₋₈ heterocycloalkene may each independently be substituted with hydroxyl (—OH), a halogen atom, C₁₋₆ alkyl or oxo (═O)).

3. The compound of claim 1, wherein the compound represented by Chemical Formula 1 is a compound represented by the following Chemical Formula 3, an optical isomer thereof, or a pharmaceutically acceptable salt thereof: 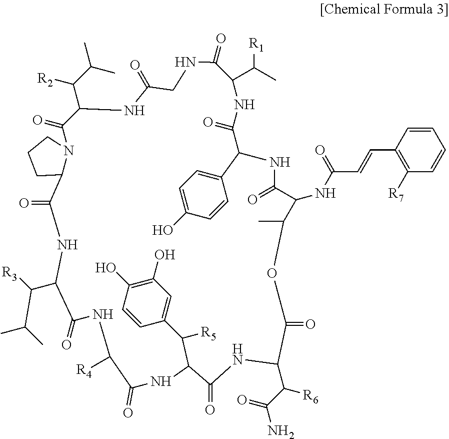
in Chemical Formula 3 above,
R₁, R₂, R₃, R₄, R₅ and R₆ are the same as or different from each other, and are each independently a hydrogen atom or hydroxyl (—OH); and
R₇ is a substituted or unsubstituted C₁₋₆ alkyl, a substituted or unsubstituted C₂₋₆ alkene, a substituted or unsubstituted C₃₋₈ heterocycloalkyl containing at least one oxygen atom (O) as a heteroatom, or a substituted or unsubstituted C₃₋₈ heterocycloalkene containing at least one oxygen atom (O) as a heteroatom (wherein at least one H of the substituted C₁₋₆ alkyl, the substituted C₂₋₆ alkene, the substituted C₃₋₈ heterocycloalkyl, or the substituted C₃₋₈ heterocycloalkene may each independently be substituted with hydroxyl (—OH), a halogen atom, C₁₋₆ alkyl or oxo (═O)).

4. The compound of claim 1, wherein
R₁, R₂, R₄ and R₅ are each independently hydroxyl (—OH); and
R₃ and R₆ are each independently a hydrogen atom.

5. The compound of claim 1, wherein
R₇ is C₁₋₄ alkyl in which at least one H is each independently substituted with hydroxyl (—OH), C₂₋₄ alkene in which at least one H is each independently substituted with hydroxyl (—OH), C₃₋₅ heterocycloalkyl containing at least one oxygen atom (O) as a heteroatom in which at least one H is each independently substituted with C₁₋₃ alkyl, oxo (═O), or both, or C₃₋₅ heterocycloalkene containing at least one oxygen atom (O) as a heteroatom in which at least one H is each independently substituted with C₁₋₃ alkyl.

6. The compound of claim 5, wherein
R₇ is C₁₋₄ alkyl substituted with at least two hydroxyls (—OH), C₂₋₄ alkene substituted with at least two hydroxyls (—OH), C₃₋₅ heterocycloalkyl containing at least one oxygen atom (O) substituted with C₁₋₃ alkyl as a heteroatom, C₃₋₅ heterocycloalkyl containing at least one oxygen atom (O) substituted with C₁₋₃ alkyl and oxo (═O) as a heteroatom, or C₃₋₅ heterocycloalkene containing one oxygen atom (O) substituted with one C₁₋₃ alkyl as a heteroatom.

7. The compound of claim 1, wherein
R₇ is propane, propene, oxirane, oxirene or dioxolane,
wherein the propane, propene, oxirane, oxirene and dioxolane may be unsubstituted, or may each independently be substituted with hydroxyl (—OH) or C₁₋₃ alky.

8. The compound of claim 1, wherein
R₇ is

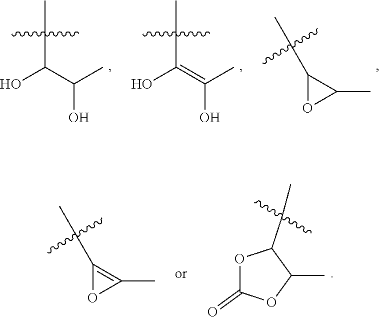

9. The compound of claim 1, wherein
the compound represented by Chemical Formula 1, an optical isomer thereof, or a pharmaceutically acceptable salt thereof is selected from the group consisting of the following Chemical Formulas 1-1, 1-2, 1-3, 1-4, 1-5 and 1-6:

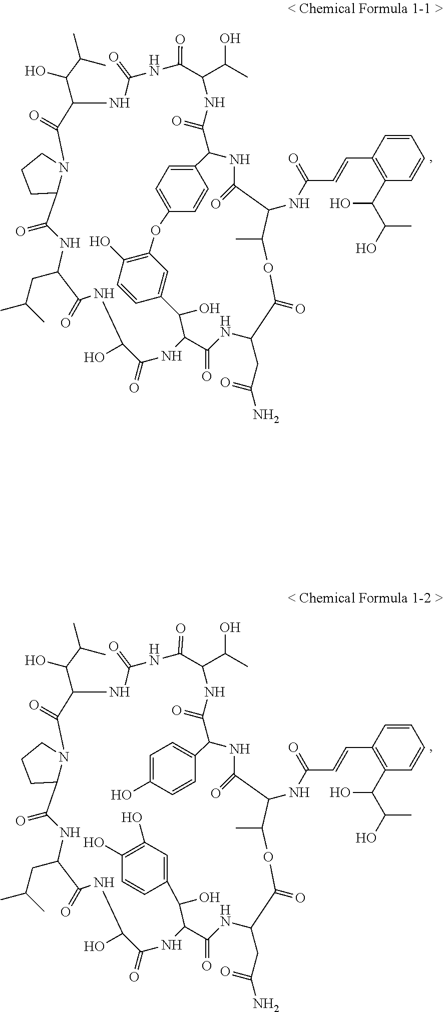 [SMILES: CC(C)CC1NC(=O)C2CCCN2C(=O)C(C(O)C(C)C)NC(=O)NC(=O)C(C(C)O)NC(=O)C2NC(=O)C(NC(=O)/C=C/c3ccccc3C(O)C(C)O)C(C)OC(=O)C(CC(N)=O)NC(=O)C(NC(=O)C(O)NC1=O)C(O)c1ccc(O)c(c1)Oc1ccc2cc1 ; CC(C)CC1NC(=O)C2CCCN2C(=O)C(C(O)C(C)C)NC(=O)NC(=O)C(C(C)O)NC(=O)C(c2ccc(O)cc2)NC(=O)C(NC(=O)/C=C/c2ccccc2C(O)C(C)O)C(C)OC(=O)C(CC(N)=O)NC(=O)C(C(O)c2ccc(O)c(O)c2)NC(=O)C(O)NC1=O]

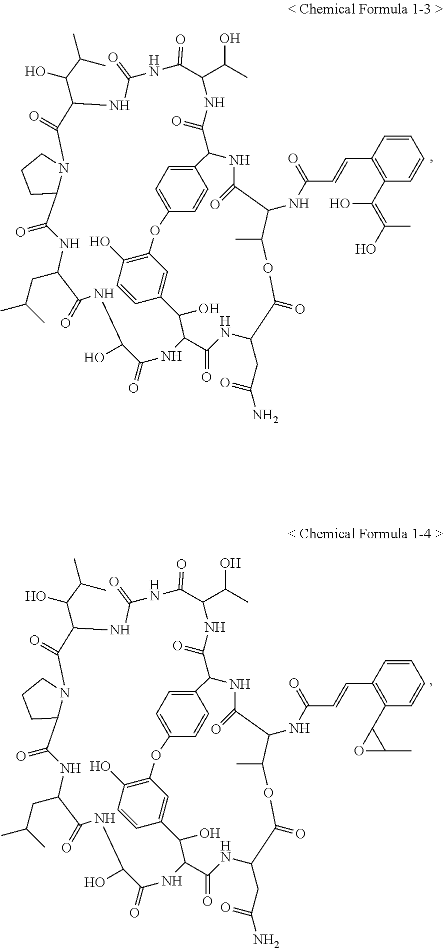 [SMILES: C/C(O)=C(/O)c1ccccc1/C=C/C(=O)NC1C(=O)NC2C(=O)NC(C(C)O)C(=O)NC(=O)NC(C(O)C(C)C)C(=O)N3CCCC3C(=O)NC(CC(C)C)C(=O)NC(O)C(=O)NC(C(=O)NC(CC(N)=O)C(=O)OC1C)C(O)c1ccc(O)c(c1)Oc1ccc2cc1 ; CC(C)CC1NC(=O)C2CCCN2C(=O)C(C(O)C(C)C)NC(=O)NC(=O)C(C(C)O)NC(=O)C2NC(=O)C(NC(=O)/C=C/c3ccccc3C3OC3C)C(C)OC(=O)C(CC(N)=O)NC(=O)C(NC(=O)C(O)NC1=O)C(O)c1ccc(O)c(c1)Oc1ccc2cc1]

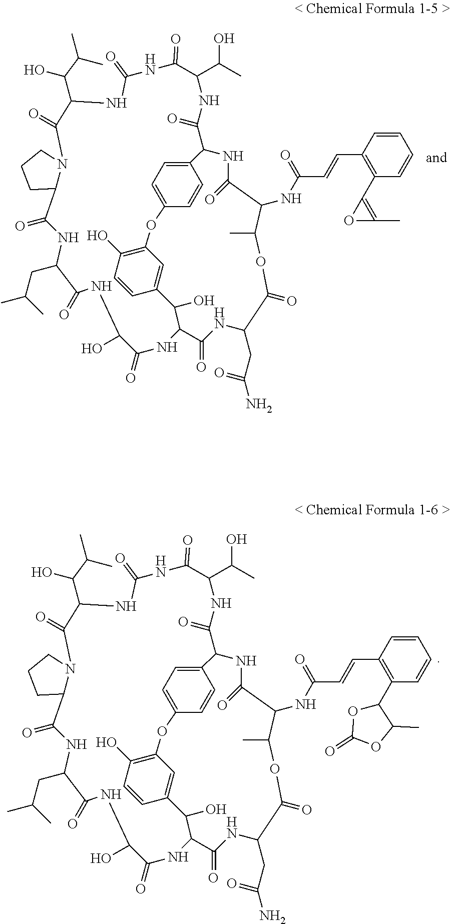 [SMILES: CC1=C(c2ccccc2/C=C/C(=O)NC2C(=O)NC3C(=O)NC(C(C)O)C(=O)NC(=O)NC(C(O)C(C)C)C(=O)N4CCCC4C(=O)NC(CC(C)C)C(=O)NC(O)C(=O)NC(C(=O)NC(CC(N)=O)C(=O)OC2C)C(O)c2ccc(O)c(c2)Oc2ccc3cc2)O1 ; CC(C)CC1NC(=O)C2CCCN2C(=O)C(C(O)C(C)C)NC(=O)NC(=O)C(C(C)O)NC(=O)C2NC(=O)C(NC(=O)/C=C/c3ccccc3C3OC(=O)OC3C)C(C)OC(=O)C(CC(N)=O)NC(=O)C(NC(=O)C(O)NC1=O)C(O)c1ccc(O)c(c1)Oc1ccc2cc1]

10. The compound of claim 1, wherein the compound, an optical isomer thereof, or a pharmaceutically acceptable salt thereof is isolated from Streptomyces rapamycinicus 17A011 KTCT 14890BP strain.

11. A pharmaceutical composition for preventing or treating Wnt/β-catenin signaling-associated diseases, comprising: the compound according to any one of claims 1 to 10, an optical isomer thereof, or a pharmaceutically acceptable salt thereof.

12. The pharmaceutical composition of claim 11, wherein the Wnt/β-catenin signaling-associated disease is at least one selected from the group consisting of neurodegenerative diseases, eye diseases, bone diseases, periodontal diseases, otosclerosis (ear sclerosis), wound healing, oral mucositis, gastrointestinal mucositis, craniofacial defects, hair loss diseases, and metabolic diseases.

13. The pharmaceutical composition of claim 12, wherein the bone disease is at least one selected from the group consisting of bone defect, osteoporosis, osteoarthrosis, osteogenesis imperfecta, bone defect, osteoporotic fracture, diabetic fracture, nonunion fracture, osteogenesis imperfecta, osteomalacia and resulting fractures, bone dysplasia, degenerative bone disease, oncolytic bone disease, Paget's disease, metabolic bone disease, leukemia, multiple myeloma, myeloma, fibrous osteodysplasia, aplastic bone disease, osteonecrosis, rickets, or malocclusion.

14. The pharmaceutical composition of claim 12, wherein the metabolic disease is at least one selected from the group consisting of obesity, dyslipidemia, fatty liver, diabetes, hyperlipidemia, hypercholesterolemia, arteriosclerosis, myocardial infarction, cerebral infarction, sarcopenia, hyperinsulinemia, and myocardial infarction.

15. The pharmaceutical composition of claim 12, wherein the neurodegenerative disease is at least one selected from the group consisting of Parkinson's disease, stroke, spinal cord injury, ischemic brain disease, epilepsy, Alzheimer's disease, dementia, depression, bipolar disorder, and schizophrenia.

16. A food composition for preventing or improving Wnt/β-catenin signaling-associated diseases, comprising: the compound according to any one of claims 1 to 10, an optical isomer thereof, or a pharmaceutically acceptable salt thereof.

17. Streptomyces rapamycinicus KTCT 14890BP.

18. A method for treating Wnt/β-catenin signaling-associated diseases comprising:
administering a therapeutically effective amount of the compound according to any one of claims 1 to 10, an optical isomer thereof, or a pharmaceutically acceptable salt thereof to a subject in need thereof.

19. Use of the compound according to any one of claims 1 to 10, an optical isomer thereof or a pharmaceutically acceptable salt thereof for the manufacture of a medicament for the treatment of Wnt/β-catenin signaling-associated diseases.

20. A pharmaceutical composition for use in the prevention or treatment of Wnt/β-catenin signaling-associated diseases, comprising: the compound according to any one of claims 1 to 10, an optical isomer thereof, or a pharmaceutically acceptable salt thereof.


SEQUENCE LISTING (9 sequences)

SEQ ID NO: 1 - DNA, 1464 bp, Artificial Sequence
  KCTC 14890BP
atccgagtttgatcatggctcaggacgaacgctggcggcgtgcttaacacatgcaagtcgaacgatgaaccggtttcggccggggattagtggcgaacgggtgagtaacacgtgggcaatctgccctgcactctgggacaagccctggaaacggggtctaataccggatatgactaccgaccgcatggtctggtggtggaaagctccggcggtgcaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcggaagcctgatgcagcgacgccgcgtgagggatgacggccttcgggttgtaaacctctttcagcagggaagaagcgcaagtgacggtacctgcagaagaagcgccggctaactacgtgccagcagccgcggtaatacgtagggcgcaagcgttgtccggaattattgggcgtaaagagctcgtaggcggcttgtcgcgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgttgtccgtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacatcggaaacatccagagatgggtgcccccttgtggtcggtgtacaggtggtgcatggctgtcgtcagctcgtgtcgtgagatgttgggttaagtcccgcaacgagcgcaacccttgttctgtgttgccagcatgcctttcggggtgatggggactcacaggagactgccggggtcaactcggaggaaggtggggacgacgtcaagtcatcatgccccttatgtcttgggctgcacacgtgctacaatggccggtacaatgagctgcgaagccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaacccttgtggagggagctgtcgaagtgggactggcgat

SEQ ID NO: 2 - DNA, 20 bp, Artificial Sequence
  mCol1a1 Forward
cgaaggcaacagtcgcttca

SEQ ID NO: 3 - DNA, 17 bp, Artificial Sequence
  mCol1a1 Reverse
cccaagttccggtgtga

SEQ ID NO: 4 - DNA, 22 bp, Artificial Sequence
  mOsteocalcin Forward
cctgagtctgacaaagccttca

SEQ ID NO: 5 - DNA, 22 bp, Artificial Sequence
  mOsteocalcin Reverse
gccggagtctgttcactacctt

SEQ ID NO: 6 - DNA, 21 bp, Artificial Sequence
  mOsterix Forward
agcgaccacttgagcaaacat

SEQ ID NO: 7 - DNA, 20 bp, Artificial Sequence
  mOsterix Reverse
gcggctgattggcttcttct

SEQ ID NO: 8 - DNA, 22 bp, Artificial Sequence
  mGAPDH Forward
catggcctccaaaggagtaaga

SEQ ID NO: 9 - DNA, 21 bp, Artificial Sequence
  mGAPDH Reverse
gagggagatgctcagtgttgg